(19) United States
(12) Patent Application Publication
(10) Pub. No.: US 2026/0000459 A1
(43) Pub. Date: Jan. 1, 2026

(54) SYSTEMS AND METHODS FOR RECONSTRUCTION OF PATIENT-SPECIFIC ANATOMICAL MODELS

(71) Applicant: LUMA Vision Limited, Dublin (IE)

(72) Inventors: Julia Hickler, Munich (DE); Lydia Neary-Zajiczek, Kerry (IE); Torsten Scheuermann, Munich (DE); Stefan Wörz, Munich (DE)

(21) Appl. No.: 19/256,756

(22) Filed: Jul. 1, 2025

Related U.S. Application Data
(60) Provisional application No. 63/666,427, filed on Jul. 1, 2024.

(51) Int. Cl. A61B 34/10; A61B 8/00; A61B 8/08; A61B 34/00; A61B 34/20; G06T 7/00; G06T 7/10

(52) U.S. Cl. CPC A61B 34/10; A61B 8/0883; A61B 8/466; A61B 34/20; A61B 34/25; G06T 7/0016; G06T 7/10; A61B 2034/105; A61B 2034/2051; A61B 2034/2061; A61B 2034/254; G06T 2200/04; G06T 2207/10136; G06T 2207/30048; G06T 2207/30101

(57) ABSTRACT

The invention relates to systems and methods for generating patient-specific 3D anatomical models of cardiac and vascular anatomy for real-time, near-real time, and/or interactive catheter and interventional tool navigation and tracking.

### CROSS-REFERENCE TO RELATED APPLICATIONS

This application claims priority to, and the benefit of, U.S. Provisional Application No. 63/666,427, filed Jul. 1, 2024, the content of which is incorporated by reference herein in its entirety.

### FIELD OF THE INVENTION

The invention generally relates to ultrasound imaging, and, more particularly, to systems and devices for providing interactive reconstruction of a patient-specific anatomical model for catheter navigation and tool tracking.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image.

For example, catheter-based endovascular ultrasound imaging technology employed within the vasculature (e.g., intravascular ultrasound (IVUS) or intracardiac echocardiography (ICE)) is commonly performed with two-dimensional (2D)-ultrasound imaging. In IVUS/ICE imaging systems, an ultrasonic transducer assembly is attached to a distal end of a catheter. The catheter is carefully maneuvered through a patient's body to an area of interest, such as within a coronary artery (for the case of IVUS), or within the right atrium (for the case of ICE). The transducer assembly transmits ultrasound waves and receives echoes from those waves. The received echoes are then converted to electrical signals and transmitted to processing equipment, in which a resulting ultrasound image of the area of interest may be displayed.

In typical ultrasound systems configured to visualize inner body regions, dynamic forces are often employed, resulting in a dynamic movement of the body regions over time. These dynamic forces and movements make it difficult to stabilize internal imaging devices and to generate consistent and accurate images. As a result, the captured images often lack the necessary quality required to prescribe appropriate treatment or therapy. Because of the dynamic forces and movements in play, internal real-time imaging is limited to small two-dimensional areas or limited three-dimensional volumetric regions respectively.

For certain treatments, such as cardiac ablation, accurately capturing a visual representation of the anatomy of interest is paramount for a successful procedure. Atrial fibrillation as well as other complex cardiac arrythmias such as atrial flutter or ventricular tachycardia are defined through irregular heartbeats caused by chaotic electrical signals in the atrial or ventricular chambers of the heart. Cardiac ablation is a common treatment approach for arrythmias, where specific regions within the heart are destroyed through ablation. In cardiac ablation, energy is applied to cardiac tissue to create scars or lesions for preventing or interrupting the transmission of abnormal electrical signals. Cardiac ablation forms an essential part of the management of cardiac arrhythmias, including supraventricular tachycardia (SVT), atrial flutter (AFL), atrial fibrillation (AF) and ventricular tachycardia (VT).

However, despite extensive utilization of imaging equipment and tools for ablation, as well as systems supporting the identification of electrically active regions of the heart, AF treatment still has a relatively low efficacy. This is due in part to limitations in tissue mapping. Thus, there is a need for improved imaging systems that allow for identifying and visualizing complex anatomy in real-time in sufficient detail to navigate precisely to target locations within the anatomy.

### SUMMARY

The present invention recognizes the limitations of currently utilized strategies for the visualization of complex anatomy, and provides systems and methods for live visualization of complex anatomy in sufficient detail for intuitive navigation to precise locations within the anatomy. Systems and methods of the invention use imaging data and various other data combined with image segmentation and registration-based or mapping-based techniques to provide for the reconstruction of a real-time or near real-time interactive patient-specific digital anatomical model.

In particular, the invention uses catheter-based ultrasound imaging data combined with, for example, three-dimensional (3D) position/orientation data, and/or pulse phase data, to reconstruct a patient-specific real-time and/or interactive digital anatomical model. The catheter-based ultrasound data may be obtained via ultrasound systems for in-body applications, such as intravascular ultrasound systems. This includes, for example, systems capable of imaging large cavities of the heart, coronary and peripheral arteries, as well as other organs such as the liver, kidneys, and the like.

Systems and methods of the invention address the clinical needs of flexible and intuitive intra-operative navigation and imaging by enabling interventional localization and tracking of catheters and tools in relation to the patient anatomy, and combining this with, for example, live intra-cardiac imaging. This gives clinical users access to intuitive anatomical orientation information within the heart and in relation to the patient's anatomy in combination with real-time imaging of the anatomy, and overcomes the limitations of fluoroscopy (poor soft tissue contrast) and current ICE systems (lack of wider context in relation to the heart).

Specific acquisition protocols guiding the clinical user allow for real-time and/or interactive model generation, for example, starting from the catheter access site (inferior vena cava) up to targets within the heart (e.g. right atrium), which enables entirely fluoroscopy-less, i.e. radiation-free, procedures. Further, systems and methods of the invention provide for advanced visualization of cardiac morphology. Providing advanced visualizations of the structure (and morphology) of cardiac chambers allows for better treatment delivery and planning of ablation paths as per the patient's cardiac anatomy. This also enables the inspection of trabeculae as well as other intracardiac structures such as tendons, chordae, and valves/leaflets. Thus, systems and methods of the invention overcome key challenges in interpreting ultrasound-based imaging data, which still mostly consists of grey-scale valued data without spatial or temporal content.

Aspects of the present invention include systems for interactive reconstruction of a digital anatomical model. The system includes a console configured to be operably associated with an ultrasound imaging device and exchange data therewith, the console comprising a hardware processor coupled to non-transitory, computer-readable memory containing instructions executable by the processor. The instructions executable by the processor cause the console to receive data associated with at least one of cardiac and vascular anatomy, wherein the data includes catheter-based ultrasound imaging data as well as for example 3D position/orientation data, and pulse phase data. The console is further configured to process and combine the received data, and reconstruct, based on the processing and combining of data, an interactive digital model of an imaged anatomy, such that the digital model encapsulates a representation of the anatomy in a spatial topology for live visualization of one or more anatomical regions of interest.

In some embodiments, the spatial topology comprises a topological map, wherein each point in the topological map represents tissue and/or one or more specific anatomical properties. Further, the ultrasound imaging data from one or more directions and/or one or more views are combined to provide the representation of the anatomy, in some embodiments. In some embodiments, the digital model is at least one of a panoramic image reconstruction, a segmented surface model, and a mesh.

In some embodiments, reconstructing the digital model comprises image-based registration and/or simultaneous localization and mapping (SLAM).

In some embodiments, the catheter-based ultrasound imaging data is received from an intracardiac echocardiogram (ICE) 2D and/or 3D catheter. In particular embodiments, the ultrasound imaging data is received from a catheter trajectory used to image the anatomical region of interest in the catheter field-of-view in a plurality of temporally overlapping segments. Further, in some embodiments, the ultrasound imaging data is received from a plurality of cylindrical fields-of-view. For example, the plurality of cylindrical fields-of-view are acquired via a rotating transducer array and/or a cylindrical folded transducer matrix, in some embodiments. In some embodiments, the ultrasound imaging data is received from a field of view whereby a motion of the catheter is perturbed from a smooth trajectory to change the viewing angle.

In particular embodiments, the pulse phase data is one or more of electrocardiogra (ECG) data, pulse oximetry data, and/or image-based intracardiac activity data. Further, in some embodiments, the received data further comprises three-dimensional (3D) position data. For example, in some embodiments, the position data comprises a spatial position and an orientation of a catheter and/or an interventional tool. Further, in some embodiments, the position data is 3D pose data comprising six degrees of freedom. The position data is obtained through one or more of electro-magnetic (EM) tracking data, impedance tracking data, image-based tracking data, and fiber optic shape sensing, in some embodiments.

In some embodiments, combining the received data comprises selecting a 3D ultrasound image in the catheter-based ultrasound imaging data and mapping the 3D ultrasound image to a physical patient coordinate system using the 3D position data such that a registration of each 3D ultrasound image in the physical patient's coordinate system is initialized. Combining the received data further comprises continuously registering subsequent 3D ultrasound images within the physical patient's coordinate system, in some embodiments. In some embodiments, the EM tracking data is combined with one or more previous registration results to predict a next 3D position. For example, the predicted next 3D position may be used for initialization of the continuous registration. Further, the continuously registered subsequent 3D ultrasound images are partly overlapping such that the continuously registered and partly overlapping subsequent 3D ultrasound images are fused into one panoramic digital model, in some embodiments. In particular embodiments, the panoramic digital model is updated sequentially over time as the catheter is moved through the one or more anatomical regions of interest. For example, the panoramic digital model may be segmented using a deep learning algorithm, in some embodiments.

In some embodiments, the continuous image registration comprises one or more of a rigid registration scheme and a deformable registration scheme. In particular, the continuous 3D ultrasound image registration comprises filtering, using pulse phase-gating, the subsequent 3D ultrasound images along the catheter's trajectory, wherein a separate digital model is generated for each cardiac phase, and wherein 3D ultrasound images of a same cardiac phase are registered against a respective previous 3D ultrasound image and/or a fused 3D image, in some embodiments. In particular embodiments, the filtered subsequent 3D ultrasound images are segmented using a supervised deep-learning algorithm. In some embodiments, an unsupervised deep-learning algorithm is used. Further, in some embodiments, reconstructing the digital model comprises fusing the segmented 3D ultrasound images using the registration results to produce a patient-specific digital 3D anatomical model. For example, in particular embodiments, the patient-specific digital 3D anatomical model is continuously updated with subsequent registration and segmentation results. The continuously updated patient-specific digital 3D anatomical model may be visualized simultaneously with the fused ultrasound image data to enable 3D navigation of the catheter, in some embodiments.

In particular embodiments, the separate 3D models for each cardiac phase are combined along the trajectory of the catheter to generate a temporal dynamic model that represents the patient-specific anatomy over time, wherein the dynamic model visualizes the heartbeat as a sequence of animated 3D models.

In some embodiments, the console is further operable to detect, localize, and segment one or more interventional tools within the 3D ultrasound images, wherein the segmented interventional tools are visualized in 3D within the digital model. For example, the detection, localization, and segmentation of the one or more interventional tools provides for contact assessment between a tool tip and a cardiac and/or a vascular wall, in some embodiments. In particular embodiments, the console is further operable to automatically detect and localize anatomical landmarks for visualization within the digital model to aid navigation of the catheter and interventional tools. Further, the catheter-based ultrasound imaging data is received from a catheter-based ultrasound transducer configured for in-body applications, in some embodiments.

In some embodiments, the interactive digital model is generated beginning from a catheter access site to one or more targets within a heart to provide an interactive visualization of patient-specific structure and/or morphology of intra-cardiac structures without the need for fluoroscopy. In particular embodiments, the intra-cardiac structures comprise one or more of trabeculae, tendons, chordae, and valves/leaflets.

Aspects of the invention provide methods for interactive reconstruction of a digital anatomical model. The methods include receiving, via a console operably associated with an ultrasound imaging device, data associated with at least one of cardiac and vascular anatomy, the data comprising catheter-based ultrasound imaging data, and pulse phase data; processing and combining, via the console, the received data; and reconstructing, based on said processing and combining of data, an interactive digital model of an imaged anatomy, wherein the digital model encapsulates a representation of the anatomy in a spatial topology for live visualization of one or more anatomical regions of interest.

In some embodiments of the methods, the spatial topology comprises a topological map, wherein each point in the topological map represents tissue and/or one or more specific anatomical properties. Further, the ultrasound imaging data from one or more directions and/or one or more views are combined to provide the representation of the anatomy, in some embodiments of the methods. In some embodiments of the method, the digital model is at least one of a panoramic image reconstruction, a segmented surface model, and a mesh.

In some embodiments of the methods, reconstructing the digital model comprises image-based registration and/or simultaneous localization and mapping (SLAM).

In some embodiments of the methods, the catheter-based ultrasound imaging data is received from an intracardiac echocardiogram (ICE) catheter comprising a 2D and/or 3D transducer. In particular embodiments of the methods, the ultrasound imaging data is received from a catheter trajectory used to image the anatomical region of interest in the catheter field-of-view in a plurality of temporally overlapping segments. Further, in some embodiments of the methods, the ultrasound imaging data is received from a plurality of cylindrical fields-of-view. For example, the plurality of cylindrical fields-of-view are acquired via a rotating transducer array and/or a cylindrical folded transducer matrix, in some embodiments of the methods. In some embodiments of the methods, the ultrasound imaging data is received from a field of view whereby a motion of the catheter is perturbed from a smooth trajectory to change the viewing angle.

In particular embodiments of the methods, the pulse phase data is one or more of electrocardiogramata, pulse oximetry data, and/or image-based intracardiac activity data. Further, in some embodiments of the methods, the received data further comprises three-dimensional (3D) position data. For example, in some embodiments of the methods, the position data comprises a spatial position and an orientation of a catheter and/or an interventional tool. Further, in some embodiments of the methods, the position data is 3D pose data comprising six degrees of freedom. The position data is obtained through one or more of electro-magnetic (EM) tracking data, impedance tracking data, image-based tracking data, and fiber optic shape sensing, in some embodiments.

In some embodiments of the methods, combining the received data comprises selecting a 3D ultrasound image in the catheter-based ultrasound imaging data and mapping the 3D ultrasound image to a physical patient coordinate system using the 3D position data such that a registration of each 3D ultrasound image in the physical patient's coordinate system is initialized. Combining the received data further comprises continuously registering subsequent 3D ultrasound images within the physical patient's coordinate system, in some embodiments of the methods. In some embodiments of the methods, the EM tracking data is combined with one or more previous registration results to predict a next 3D position. For example, the predicted next 3D position may be used for initialization of the continuous registration. Further, the continuously registered subsequent 3D ultrasound images are partly overlapping such that the continuously registered and partly overlapping subsequent 3D ultrasound images are fused into one panoramic digital model, in some embodiments of the methods. In particular embodiments of the methods, the panoramic digital model is updated sequentially over time as the catheter is moved through the one or more anatomical regions of interest. For example, the panoramic digital model may be segmented using a deep learning algorithm, in some embodiments of the methods.

In some embodiments of the methods, the continuous image registration comprises one or more of a rigid registration scheme and a deformable registration scheme. In particular, the continuous 3D ultrasound image registration comprises filtering, using pulse phase-gating, the subsequent 3D ultrasound images along the catheter's trajectory, wherein a separate digital model is generated for each cardiac phase, and wherein 3D ultrasound images of a same cardiac phase are registered against a respective previous 3D ultrasound image and/or a fused 3D image, in some embodiments of the methods. In particular embodiments of the methods, the filtered subsequent 3D ultrasound images are segmented using a supervised deep-learning algorithm. In some embodiments, an unsupervised deep-learning algorithm is used. Further, in some embodiments of the methods, reconstructing the digital model comprises fusing the segmented 3D ultrasound images using the registration results to produce a patient-specific digital 3D anatomical model. For example, in particular embodiments of the methods, the patient-specific digital 3D anatomical model is continuously updated with subsequent registration and segmentation results. The continuously updated patient-specific digital 3D anatomical model may be visualized simultaneously with the fused ultrasound image data to enable 3D navigation of the catheter, in some embodiments of the methods.

In particular embodiments of the methods, the separate 3D models for each cardiac phase are combined along the trajectory of the catheter to generate a temporal dynamic model that represents the patient-specific anatomy over time, wherein the dynamic model visualizes the heartbeat as a sequence of animated 3D models.

In some embodiments of the methods, the console is further operable to detect, localize, and segment one or more interventional tools within the 3D ultrasound images, wherein the segmented interventional tools are visualized in 3D within the digital model. For example, the detection, localization, and segmentation of the one or more interventional tools provides for contact assessment between a tool tip and a cardiac and/or a vascular wall, in some embodiments of the methods. In particular embodiments of the methods, the console is further operable to automatically detect and localize anatomical landmarks for visualization within the digital model to aid navigation of the catheter and interventional tools. Further, the catheter-based ultrasound imaging data is received from a catheter-based ultrasound comprising an endovascular and/or intravascular ultrasound configured for in-body applications, in some embodiments of the methods.

In some embodiments of the methods, the interactive digital model is generated beginning from a catheter access site to one or more targets within a heart to provide an interactive visualization of patient-specific structure and/or morphology of intra-cardiac structures without the need for fluoroscopy. In particular embodiments of the methods, the intra-cardiac structures comprise one or more of trabeculae, tendons, chordae, valves/leaflets.

Aspects of the invention provide systems for interactive catheter and/or interventional tool tracking. The system includes a console configured to be operably associated with an ultrasound imaging device and exchange data therewith, the console comprising a hardware processor coupled to non-transitory, computer-readable memory containing instructions executable by the processor to cause the console to receive data associated with at least one of cardiac and vascular anatomy, the data comprising catheter-based ultrasound imaging data, and pulse phase data, process and combine the received data; and reconstruct, based on said processing and combining of data, an interactive digital model of a catheter and/or interventional tool in an imaged anatomy, wherein the digital model encapsulates a representation of the catheter and/or interventional tool in a spatial anatomical topology for live visualization of the catheter and/or interventional tool in one or more anatomical regions of interest.

In some embodiments, the spatial topology comprises a topological map, wherein each point in the topological map represents tissue and/or one or more specific anatomical properties. Further, the ultrasound imaging data from one or more directions and/or one or more views are combined to provide the representation of the anatomy, in some embodiments. In some embodiments, the digital model is at least one of a panoramic image reconstruction, a segmented surface model, and a mesh.

In some embodiments, reconstructing the digital model comprises image-based registration and/or simultaneous localization and mapping (SLAM).

In some embodiments, the catheter-based ultrasound imaging data is received from an intracardiac echocardiogram (ICE) 2D and/or 3D catheter. In particular embodiments, the ultrasound imaging data is received from a catheter trajectory used to image the anatomical region of interest in the catheter field-of-view in a plurality of temporally overlapping segments.

Further, in some embodiments, the ultrasound imaging data is received from a plurality of cylindrical fields-of-view. For example, the plurality of cylindrical fields-of-view are acquired via a rotating transducer array and/or a cylindrical folded transducer matrix, in some embodiments. In some embodiments, the ultrasound imaging data is received from a field of view whereby a motion of the catheter is perturbed from a smooth trajectory to change the viewing angle.

In particular embodiments, the pulse phase data is one or more of electrocardiogra (ECG) data, pulse oximetry data, and/or image-based intracardiac activity data. Further, in some embodiments, the received data further comprises three-dimensional (3D) position data. For example, in some embodiments, the position data comprises a spatial position and an orientation of a catheter and/or an interventional tool. Further, in some embodiments, the position data is 3D pose data comprising six degrees of freedom, (i.e. translation and rotation around the x, y, and z axes). The position data is obtained through one or more of electro-magnetic (EM) tracking data, impedance tracking data, image-based tracking data, and fiber optic shape sensing, in some embodiments.

In some embodiments, combining the received data comprises selecting a 3D ultrasound image in the catheter-based ultrasound imaging data and mapping the 3D ultrasound image to a physical patient coordinate system using the 3D position data such that a registration of each 3D ultrasound image in the physical patient's coordinate system is initialized. Combining the received data further comprises continuously registering subsequent 3D ultrasound images within the physical patient's coordinate system, in some embodiments. In some embodiments, the EM tracking data is combined with one or more previous registration results to predict a next 3D position. For example, the predicted next 3D position may be used for initialization of the continuous registration. Further, the continuously registered subsequent 3D ultrasound images are partly overlapping such that the continuously registered and partly overlapping subsequent 3D ultrasound images are fused into one panoramic digital model, in some embodiments. In particular embodiments, the panoramic digital model is updated sequentially over time as the catheter is moved through the one or more anatomical regions of interest. For example, the panoramic digital model may be segmented using a deep learning algorithm, in some embodiments.

In some embodiments, the continuous image registration comprises one or more of a rigid registration scheme and a deformable registration scheme. In particular, the continuous 3D ultrasound image registration comprises filtering, using pulse phase-gating, the subsequent 3D ultrasound images along the catheter's trajectory, wherein a separate digital model is generated for each cardiac phase, and wherein 3D ultrasound images of a same cardiac phase are registered against a respective previous 3D ultrasound image and/or a fused 3D image, in some embodiments. In particular embodiments, the filtered subsequent 3D ultrasound images are segmented using a supervised deep-learning algorithm. In some embodiments, an unsupervised deep-learning algorithm is used. Further, in some embodiments, reconstructing the digital model comprises fusing the segmented 3D ultrasound images using the registration results to produce a patient-specific digital 3D anatomical model. For example, in particular embodiments, the patient-specific digital 3D anatomical model is continuously updated with subsequent registration and segmentation results. The continuously updated patient-specific digital 3D anatomical model may be visualized simultaneously with the fused ultrasound image data to enable 3D navigation of the catheter, in some embodiments.

In particular embodiments, the separate 3D models for each cardiac phase are combined along the trajectory of the catheter to generate a temporal dynamic model that represents the patient-specific anatomy over time, wherein the dynamic model visualizes the heartbeat as a sequence of animated 3D models.

In some embodiments, the console is further operable to detect, localize, and segment one or more interventional tools within the 3D ultrasound images, wherein the segmented interventional tools are visualized in 3D within the digital model. For example, the detection, localization, and segmentation of the one or more interventional tools provides for contact assessment between a tool tip and a cardiac and/or a vascular wall, in some embodiments. In particular embodiments, the console is further operable to automatically detect and localize anatomical landmarks for visualization within the digital model to aid navigation of the catheter and interventional tools. Further, the catheter-based ultrasound imaging data is received from a catheter-based ultrasound comprising an endovascular and/or intravascular ultrasound configured for in-body applications, in some embodiments.

In some embodiments, the interactive digital model is generated beginning from a catheter access site to one or more targets within a heart to provide an interactive visualization of patient-specific structure and/or morphology of intra-cardiac structures without the need for fluoroscopy. In particular embodiments, the intra-cardiac structures comprise one or more of trabeculae, tendons, chordae, and valves/leaflets.

### BRIEF DESCRIPTION OF THE DRAWINGS

FIGS. 1A, 1B, and 1C illustrate the state of current imaging modalities available for interventional cardiology.

FIG. 2A and FIG. 2B are diagrammatic illustrations of an exemplary ultrasound system for providing catheter-based ultrasound imaging data specific to a patient.

FIG. 3 is a perspective view of an imaging catheter with which systems of the invention may be coupled.

FIG. 4 illustrates one embodiment of systems of the invention.

FIG. 5 illustrates the orbital field-of-view around one embodiment of a catheter utilized in systems of the invention.

FIG. 6 illustrates a 4D heart model that may be composed using systems of the invention.

FIG. 7 illustrates a block diagram of a method for interactive reconstruction of a digital anatomical model according to one embodiment.

FIG. 8A illustrates a blending method according to one embodiment of the invention.

FIG. 8B illustrates visual results of the blending methods according to one embodiment of the invention as compared to average intensity blending.

FIG. 9 illustrates a reconstruction of patient specific cardiac anatomy according to one embodiment of the systems and methods of the invention.

FIGS. 10-13 illustrate panoramic segmentation according to some embodiments of the systems and methods of the invention.

### DETAILED DESCRIPTION

The present invention recognizes the limitations of currently utilized strategies, such as fluoroscopy, 2D and 3D catheter-based ultrasound imaging, and electroanatomical mapping (EAM), for the visualization of complex anatomy, and provides systems and methods for live visualization of complex anatomy in sufficient detail for intuitive navigation to precise locations within the anatomy.

Systems and methods of the invention use imaging data and various other data combined with image segmentation and registration-based or mapping-based techniques for real-time or near-real-time interactive reconstruction of a patient-specific digital anatomical model. In particular, the invention uses catheter-based ultrasound imaging data combined with, for example, three-dimensional (3D) position/orientation data, and/or pulse phase data, to reconstruct a patient-specific real-time and/or interactive digital anatomical model. The catheter-based ultrasound data may be obtained via endovascular and intravascular ultrasound systems for in-body applications. This includes, for example, systems capable of imaging large cavities of the heart, coronary and peripheral arteries, as well as other organs such as the liver, kidneys, and the like.

In particular embodiments, systems and methods of the invention may use a 3D intracardiac echocardiography (ICE) catheter to acquire ultra-fast 4D ultrasound image data of a considered cardiac and/or vascular anatomy. Information on cardiac pulse phase and/or 3D position data of the ICE catheter may be utilized to map the ultrasound image data to a patient coordinate system. The systems also provide for integrating 3D position data of interventional tools for live visualization of the interventional tools within the anatomy.

As is disclosed in more detail herein, the 3D position data may be obtained through, for example, electromagnetic (EM) tracking, impedance tracking, image-based tracking, fiber optic shape sensing, or a combination of these or other modalities. Cardiac pulse phase data may be obtained via electrocardiogramalse oximetry, intracardiac activity data from catheters or image-based methods. As such, the invention provides systems and methods for interactive imaging and flexible and intuitive intra-operation navigation that enables interventional localization and tracking of catheters and tools in relation to the patient anatomy. This allows for real-time and/or interactive model generation of complex anatomy, for example, interactive generation of a patient-specific model of endocardial and/or myocardial anatomy, starting from the catheter access site, e.g. inferior vena cava, and up to targets within the heart (e.g. right atrium).

Based on the limitations of current systems, and the need for intuitive and efficient navigation for operators, the present invention provides systems and methods for imaging complex anatomy, such as cardiac structures, with real-time, near-real-time, and/or interactive anatomical context. Thus, in particular embodiments, the systems and methods of the invention provide for detailed live/interactive inspection of structures of interest for interventionalists performing complex catheter-based procedures.

As is disclosed in more detail herein, features of the systems and methods of the invention include the use of an appropriate trajectory of the catheter to cover the considered anatomical region in its field-of-view (FOV) in temporally overlapping segments, such that no complex steering and no tissue contact are required. Systems and methods of the invention provide for rapid processing as compared with electroanatomical (EAM) systems, and provide for more accurate anatomical models than EAM systems, approaching the quality of models obtained from pre-operative computed tomography angiography (CTA) data. The systems and methods of the invention provide for generating an extended dynamic model by considering, in part, different phases of the heart cycle, thus generating a combination of different models for each ECG-gated phase. Importantly, systems and methods of the invention do not require fluoroscopy methods, and thus reduce the radiation dosage for operator and patient alike.

In exemplary embodiments, the systems and methods of the invention address the clinical needs of flexible and intuitive intra-operative navigation and imaging by enabling interventional localization and tracking of catheters and tools in relation to the patient anatomy, and combining this with live intra-cardiac imaging. This gives clinical users access to intuitive anatomical orientation information within the heart and in relation to the patient's anatomy in combination with real-time imaging of the anatomy, and overcomes the limitations of fluoroscopy (poor soft tissue contrast) and current ICE systems (lack of wider context in relation to the heart).

Systems and methods of the invention also address the clinical need for real-time, near-real-time, and/or interactive digital reconstruction of patient anatomy. Specific acquisition protocols guiding the clinical user allow for real-time model generation, for example, starting from the catheter access site (inferior vena cava) up to targets within the heart (e.g. right atrium), which enables entirely fluoroscopy-less (i.e. radiation-free) procedures. Further, systems and methods of the invention provide for advanced visualization of cardiac morphology. Providing advanced visualizations of the structure (and morphology) of cardiac chambers allows for better treatment delivery and planning of ablation paths as per the patient's cardiac anatomy. This enables the inspection of trabeculae as well as other intracardiac structures such as tendons, chordae, and valves/leaflets. Thus, the systems and methods of the invention overcome key challenges in interpreting ultrasound-based imaging data, which still mostly consists of grey-scale valued data unchanged from its original introduction as an interventional imaging modality 75 years ago.

### Overview

The invention provides systems and methods for interactive, live, and/or real-time reconstruction of a patient-specific digital anatomical model, for example, interactive reconstruction of complex cardiac and vascular anatomy.

Atrial fibrillation, as well as other complex cardiac arrythmias such as atrial flutter or ventricular tachycardia, may be defined through irregular heartbeats caused by chaotic electrical signals in atrial or ventricular chambers of the heart. Cardiac ablation is a common treatment approach for arrhythmias. Catheter ablation is a treatment in which energy is applied to cardiac tissue to create scars or lesions for preventing or interrupting the transmission of abnormal electrical signals. In catheter ablation, specific regions within the heart are destroyed resulting in electrical isolation of these regions to prevent a propagation of electrical signals causing the arrhythmia.

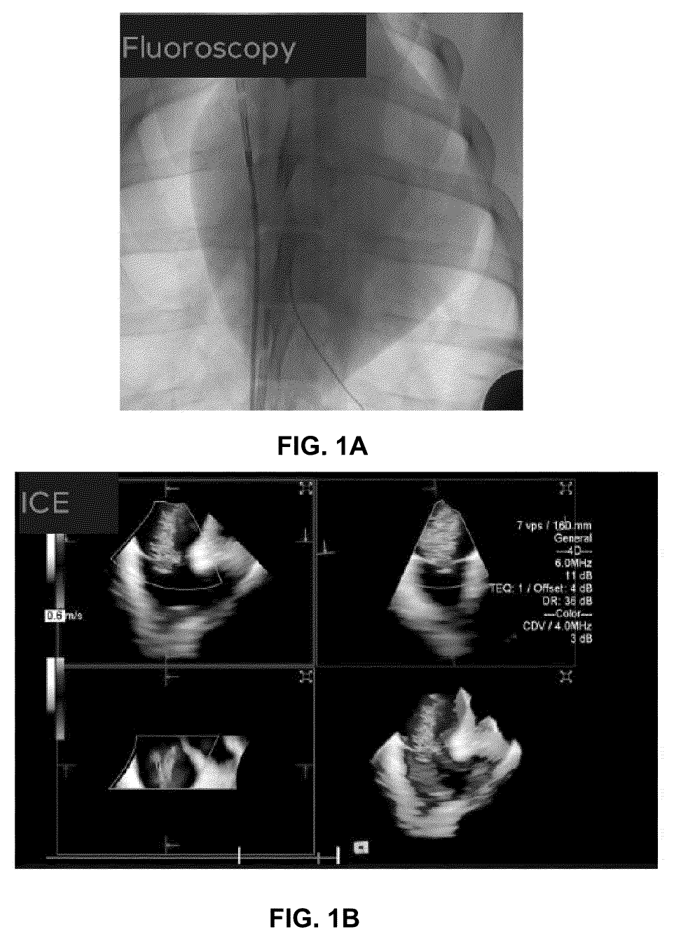

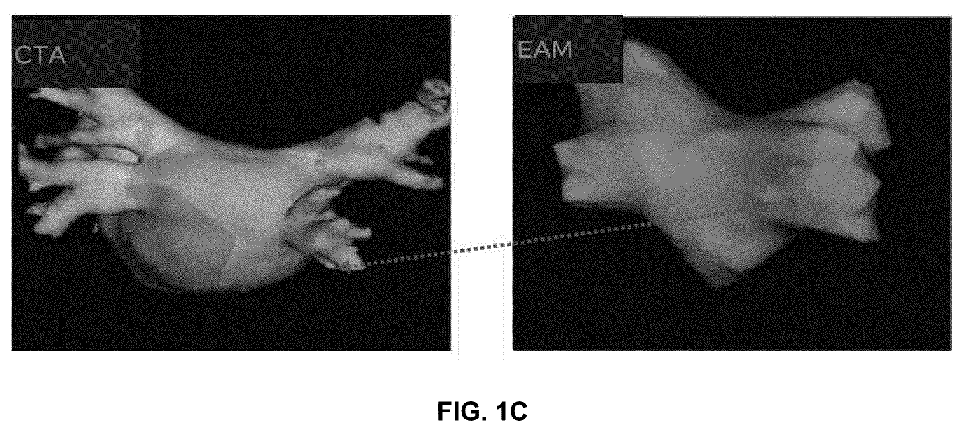

Despite the extensive utilization of imaging equipment and tools for ablation, as well as systems supporting the identification of electrically active regions within the heart (e.g. electroanatomical mapping systems), conventional imaging systems do not allow a clinical user to understand the complex endocardial and myocardial anatomy live and in sufficient detail such that the user can navigate intuitively to precisely target locations within the heart. The invention solves these problems by providing imaging systems and methods for the reconstruction of patient-specific anatomy that allows for easy to use and understand (i.e. intuitive) navigation. FIGS. 1A, 1B, and 1C illustrate the state of current imaging modalities available for interventional cardiology. Current strategies for imaging include fluoroscopy, intracardiac echocardiography (ICE), and electroanatomical mapping (EAM). The present invention addresses the drawbacks of these strategies, and provides for interventional localization and tracking of catheters and tools in relation to the patient anatomy in combination with live/interactive ultrasound imaging, such as intra-cardiac imaging.

FIG. 1A shows an image generated via fluoroscopy, an x-ray based modality.

Fluoroscopy, i.e. 2D projections generated on demand via C-Arm X-ray systems, is used in almost all interventions as it provides an “anatomical context” as well as clinical familiarity due to its inclusion in electrophysiology training. As such, while it suffers from poor soft tissue contrast, it is still the cornerstone of procedures, particularly in single shot ablations because of a lack of a better alternative. However, fluoroscopy is a radiation-based modality and the necessity of cumbersome personal protective equipment (e.g. lead vests) can lead to chronic neck or back injuries for many clinicians as well as cataract and other ocular diseases for interventional staff in the long term.

FIG. 1B shows images generated via intracardiac echocardiography (ICE) using ultrasound waves for imaging. ICE, i.e. 2D and 3D catheter-based ultrasound imaging, provides high soft tissue contrast and enables real-time, close-up imaging of cardiac structures. ICE is a catheter-based form of echocardiography that gathers images from within the heart, rather than by gathering images of the heart by sending sound waves through the chest wall. ICE is used as a standard step in many procedures for transseptal crossings, and generally in procedural steps where dynamic anatomical information may be required in real-time. However, displayed gray-scale ultrasound data is challenging to interpret as it lacks the anatomical context of surrounding structures and is susceptible to speckle, clutter, and other artefacts inherent to ultrasound as an imaging modality. This is rendered more complex as probes/catheters require manual guidance while imaging the anatomy, requiring operators to have an anatomical context, hence its general use is in tandem with fluoroscopy to provide a wider context.

FIG. 1C right panel shows an image generated by electroanatomical mapping (EAM).

EAM systems allow for the reconstruction of a shell of a cardiac chamber (Left Atrium in this example) but provide limited accuracy and spatial detail as compared to a CTA (computed tomography angiography) image, as shown in FIG. 1C left panel. EAM systems are not imaging systems per se, but provide anatomical navigation by taking advantage of the presence of electrical activity inside the heart. These systems reconstruct an anatomical shell from a number of catheter positions endocardially (on the inner heart surface), where thousands of catheter positions are recorded throughout several minutes in order to reconstruct a model from a cloud of points. Data may be recorded for so-called “mapping” catheters, where the tip position may be acquired through a tracking technology (e.g. EM tracking) and combined with electrical mapping information (voltages acquired at one to many electrodes). This reconstruction provides a combined electrical and an anatomical model (hence EAM), but requires a reconstruction from a series of point acquisitions which inherently are not real-time and suffer from intrinsic errors.

The present invention overcomes the aforementioned drawbacks. Systems and methods of the invention generate a reliable and anatomically correct patient-specific 3D digital anatomical model and interventional tools within the model. As such, a model may be generated during procedures, such as intracardiac procedures, that can be used in real-time, near real-time and/or interactively. For intracardiac procedures, the model may be generated based on received ultrasound image data obtained from an ICE catheter and combined with, for example, electrocardiogramata and 3D position data, such as EM tracking data. This provides real-time 3D localization and tracking of the ICE catheter and interventional tools with the generated model, enabling navigation of the ICE catheter, and tool tracking and navigation including contact assessment. The systems of the invention provide for faster processing as compared with EAM systems, and much more accurate models. Importantly, systems and methods of the invention require no fluoroscopy, i.e. the systems and methods of the invention provide for radiation-free imaging.

### Ultrasound Imaging System

As is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). In an ultrasound exam, the imaging device, (i.e. the transducer, probe, or transducer probe) may be placed directly on the skin or inside a body opening (e.g. endovascular ultrasound, intravascular ultrasound, intracardiac echocardiography). The final quality of the image obtained through ultrasound scanning may be limited to the technical specifications of the equipment, the propagation of ultrasonic waves through the tissue analyzed, and the method used to reconstruct the images.

Systems of the invention may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein. The data may be processed using imaging protocols to extract anatomical and functional information, and tissue characteristics as disclosed in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein.

Systems of the invention are configured to receive three-dimensional (3D) ultrasound image data from an imaging device. In some embodiments, the invention provides for reconstruction of a patient-specific anatomical model for use in minimally invasive procedures in the vasculature. Accordingly, ultrafast ultrasound imaging techniques, such as plane wave or diverging wave imaging, may be required to enable imaging within the constraints of the application, particularly for intravascular and/or intracardiac tissue assessment and analysis. These constraints could be posed due to the high temporal update rate as required for effects observed in visualization and tissue characterization, where plane and diverging wave methods enable high imaging rates, commonly also referred to ultrafast imaging approaches. Systems and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques. Thus, the three-dimensional (3D) ultrasound image data received by systems of the invention may be real-time 3D ultrasound data. For example, the data may be full circumferential, 3D image data.

While exemplary embodiments describe ultrasound imaging data received from 3D ICE catheters, catheter-based ultrasound imaging as described herein is not limiting. Imaging data may be received from catheter-based ultrasound systems that may include endovascular and/or intravascular ultrasound for in-body applications. This may include systems and imaging data for imaging in large cavities of the heart, coronary and peripheral arteries, as well as other organs such as liver, kidney, and the like.

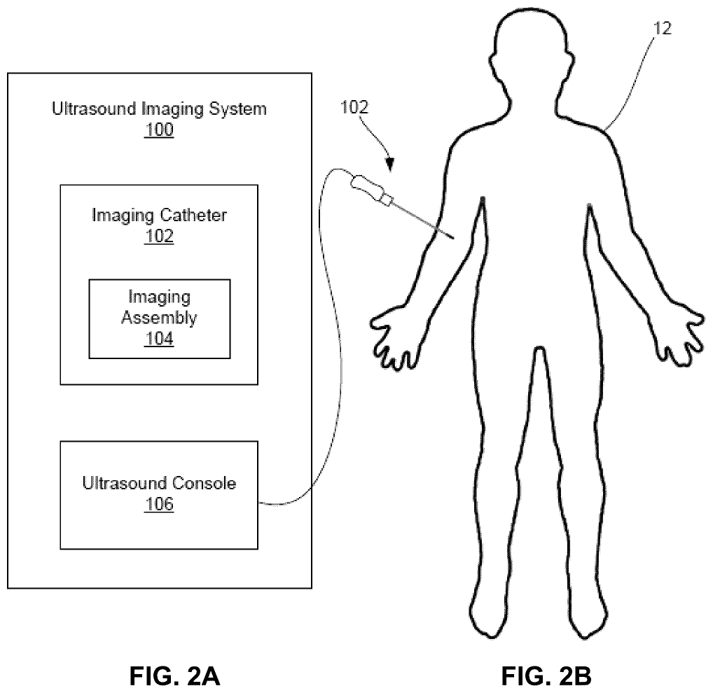

FIG. 2A and FIG. 2B are diagrammatic illustrations of an exemplary ultrasound system 100 for providing comprising catheter-based ultrasound imaging data specific to a patient 12. The systems 100 may include an imaging device equipped with an imaging assembly 104 and a console 106 to which the imaging device is to be connected. The imaging device may be an imaging catheter 102. Accordingly, systems and methods for interactive reconstruction of patient-specific digital model of cardiac and/or vascular anatomy may use a four-dimensional (4D) Intracardiac echocardiogram (ICE) system that captures the anatomy of interest. In specific embodiments of the systems, the ultrasound imaging device comprises a 4D catheter-based ultrasound imaging device. In this context, 4D may mean 3D imaging plus a time component.

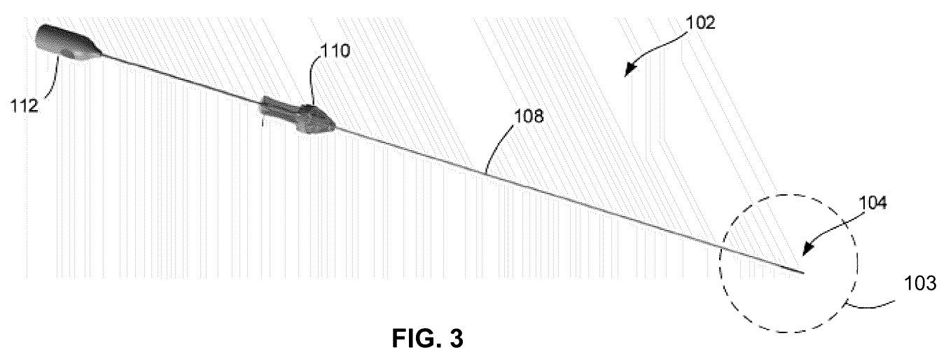

FIG. 3 is a perspective view of an imaging catheter 102 with which systems of the invention may be coupled. The catheter 102 may include a catheter body 108, including proximal and distal portions. The imaging assembly 104 may be provided at the distal portion 103, for example, generally defining a distal end of an imaging catheter. A handle 110 may be operably associated with the catheter body 108 and allow for an operator (i.e., surgeon or other medical professional) to manipulate and advance the imaging assembly 104 and the catheter body 108 to a desired target site within the patient's vasculature. The handle 110 may include user-operable inputs for controlling various features and functions of the imaging assembly 104. An interface member 112 may be provided at a proximal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

The console may include a hardware processor coupled to non-transitory, computer-readable memory, such that the computer-readable memory contains instructions executable by the processor to cause the console to receive a plurality of data, process and combine the data, and reconstruct, based on the processing and combining of the data, an interactive digital model of an imaged anatomy.

The console may be operably coupled to the imaging device and may generally control operation of the transducer probe i.e., transmission of sound waves from the probe. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which may be stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

As disclosed in more detail herein, the console may be operable to receive available ultrasound imaging data, pulse phase data, and/or position data, to process and combine the received data, and to reconstruct, based on the processing and combining of the data, an interactive digital model of an imaged anatomy. The digital model encapsulates a representation of the anatomy in a spatial topology for live, real-time, and/or interactive visualization of one or more anatomical regions of interest.

### Systems for Real-Time Reconstruction of a Patient-Specific Anatomical Model

Aspects of the invention provide systems for interactive reconstruction of a digital anatomical model. The systems includes a console configured to be operably associated with an ultrasound imaging device and to exchange data therewith. The console may comprise a hardware processor coupled to non-transitory, computer-readable memory containing instructions executable by the processor to cause the console to receive data associated with a complex anatomy. In some embodiments, the complex anatomy may be at least one of cardiac and vascular anatomy, such that the data comprises catheter-based ultrasound imaging data and pulse phase data.

The console may be operable to process and combine the received data, and reconstruct, based on the processing and combining of data, an interactive digital model of an imaged anatomy. The digital model encapsulates a representation of the anatomy in a spatial topology for live visualization of one or more anatomical regions of interest.

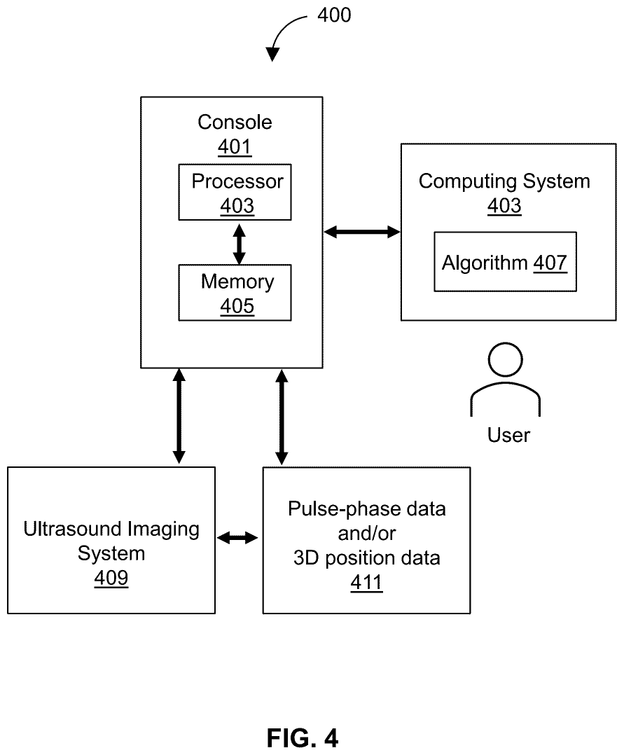

FIG. 4 illustrates one embodiment of systems 400 of the invention. For example, some embodiments, the systems 400 may include a console 401 in active communication with a computing system 403 configured to communicate across a network. The console 401 may be configured to be operably associated with an ultrasound imaging device 409, as well as with other devices and/or means for receiving data 411, and to exchange data therewith. The console may comprise a hardware processor 403 coupled to non-transitory, computer-readable memory 405 containing instructions executable by the processor to cause the console to receive data associated with at least one of cardiac and vascular anatomy the data comprising catheter-based ultrasound imaging data, and, in some embodiments, pulse phase data and/or 3D position data 411. The console may be operable to process and combine the received data, and reconstruct, based on said processing and combining of data, an interactive digital model of an imaged anatomy, wherein the digital model encapsulates a representation of the anatomy in a spatial topology for live visualization of one or more anatomical regions of interest.

The computing system 403 or computing device may include one or more processors and memory, as well as an input/output mechanism (i.e., a keyboard, knobs, scroll wheels, or the like) with which a user can interact so as to operate the console, including making adjustments to the ultrasound imaging system 409, saving images, initialization, continuous 3D image registration, filtering, optimization, 3D fusion, and panoramic image reconstruction.

The computing system 403 may include a computer program comprising one or more algorithms 407 for image registration, filtering, segmentation, optimization, and reconstruction of the digital anatomical model. For example, the algorithm 407 may be part of a computer program executable by the computing system 403 and in communication with the console 401 of the systems 400. The systems may be in communication with the imaging device 409 to receive 3D ultrasound image data from the imaging device 409.

As disclosed in detail herein, imaging protocols and algorithms may be used to dynamically reconstruct properties of the anatomy. The systems may include one or more algorithms for dynamically reconstructing multiple images from the 3D image data to provide a 3D visualization of the anatomical region of interest and targeted tissue site.

### Received Data

The systems of the invention receive, process, and combine real-time catheter-based ultrasound imaging data with other data such as pulse-phase data and/or 3D pose data (position and orientation data) to reconstruct a representation of the digital anatomy. Systems of the invention generate a reliable and anatomically correct patient-specific 3D digital anatomical model.

### Ultrasound Imaging Data:

Systems of the invention are configured to receive and process ultrasound imaging data. In some embodiments, the ultrasound imaging data may be intracardiac ultrasound image data. As such, the systems may use available 3D ICE catheters for acquiring ultra-fast 4D ultrasound image data of a considered cardiac and/or vascular anatomy for processing and combining with other data to generate the reconstructed digital anatomical model.

In some embodiments, systems of the invention utilize multiple fields-of-view, e.g. cylindrical fields-of-view acquired with a rotating transducer array or a cylindrical folded transducer matrix. Thus, cylindrical/360-degree imaging may be applied to provide an extended field-of view (FOV).

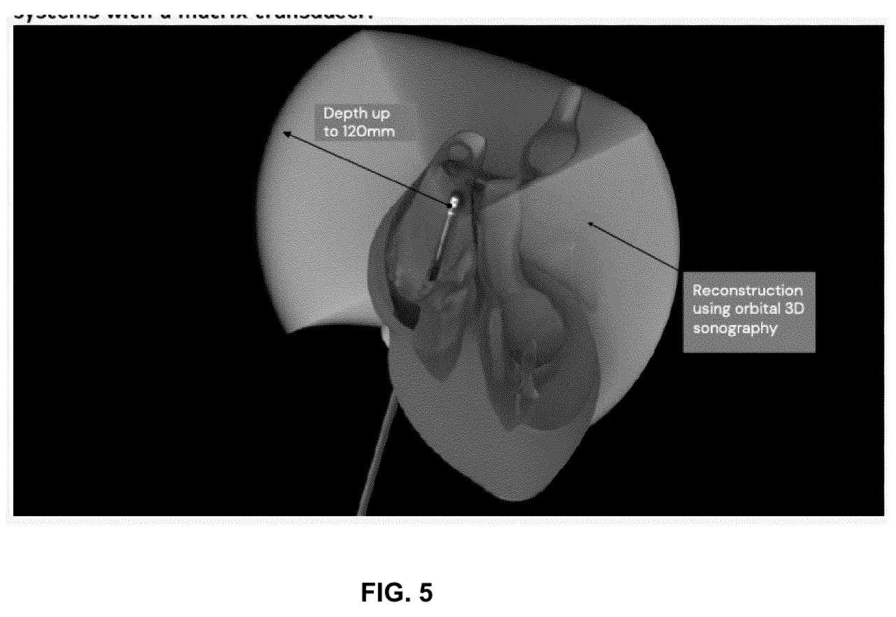

FIG. 5 illustrates the orbital field-of-view around one embodiment of a catheter utilized in systems of the invention, which may be different than the planar field-of-view of conventional ICE catheters with a matrix transducer. 3D ICE catheters with forward and with sideways transducer arrays are suitable as long as the field-of-view is sufficiently large, to provide for panoramic reconstruction. For example, catheters facing sideways may be used as these catheters also cover the forward direction to some degree due to the opening angle of the ultrasound beam. Any field-of view implementations may be used to achieve imaging of the whole vascular geometry and its surroundings for each volume such that imaging may be 360 degrees around the catheter. In some embodiments, the ultrasound imaging data may be received from a plurality of cylindrical fields-of-view. For example, in some embodiments, the plurality of cylindrical fields of view are acquired via a rotating transducer array and/or a cylindrical folded transducer matrix.

Generally, a whole considered anatomical region is not covered by a single 3D ultrasound image. Therefore, in some embodiments, systems of the invention provide for selecting an appropriate trajectory of the ICE catheter such that the whole region may be covered over time by multiple partly overlapping 3D images. Systems of the invention may provide a trajectory starting from the catheter access site up to targets of interest, for example targets within the heart. Systems of the invention provide for reconstructing a digital anatomy from different acquisitions. A clinically important protocol is a trajectory starting from the catheter access site up to the targets within the heart, which enables procedures requiring no fluoroscopy. This is particularly useful in the reconstruction of the intracardiac digital anatomy. The method includes acquiring data during the advancement of the catheter from the access site up into the right atrium. Further data may be acquired during pull back (retraction) from the superior vena cava into the inferior vena cava for reconstruction of a right atrial map and left atrial map. Data may be collected from a pullback from the left sided pulmonary veins into the septal wall for reconstructing a high resolution left atrial map.

To increase the field-of-view, the catheter's motion may be perturbed slightly from a smooth trajectory in a defined way. For example, the catheter tip may be disturbed or “wobbled” using a built-in steering mechanism, which changes the viewing angle. By doing so, the image contrast of interfaces that are not well aligned with the ultrasound wave front in the smooth trajectory is improved. Thus, in some embodiments, the ultrasound imaging data may be received from a field of view whereby a motion of the catheter may be perturbed from a smooth trajectory to change the viewing angle. Further, in some embodiments, the speed of the ICE catheter's trajectory may be limited by the field-of-view of the ultrasound image data such that subsequent phase-gated, e.g. ECG-gated, 3D images have sufficient overlap.

### Pulse Phase Data:

Information on cardiac pulse phase may also be received, processed, and combined with the ultrasound imaging data to generate the reconstructed digital anatomical model. Pulse phase data may be received from other systems for pulse phase information such as pulse oximetry, intracardiac activity data from one or more catheters, or image-based methods. In some embodiments, the pulse phase data may be one or more of electrocardiogramata, pulse oximetry data, and/or image-based intracardiac activity data. For example, pulse phase data may be received via ECG. The pulse-phase data, such as ECG data, may be used to filter the cardiac phases.

### 3D Position and Orientation Data:

Systems of the invention provide for the real-time 3D localization and tracking of the catheter, for example an ICE catheter, and interventional tools within the patient coordinate system. Systems of the invention may receive 3D position and orientation data of the catheter, and process and combine this data with the ultrasound imaging data and/or the pulse phase data for reconstruction of the digital anatomical model. It is noted however, that in some embodiments, reconstruction of the digital anatomical model is possible without measured 3D position data. In this case the 3D position may be inferred from the image data. The 3D position and orientation data may be referred to as 3D pose data. 3D pose data may be 6 degree-of-freedom tracking including position and orientation. The 3D pose data may comprise a position and orientation in 3D space.

Tracking data may be used by the console to map the ultrasound image data to a patient coordinate system.

The 3D pose data may be obtained through electromagnetic (EM) tracking, impedance tracking, image-based tracking, fiber-optic shape sensing, and/or a combination of these modalities. In some embodiments, EM tracking data may be used. Electromagnetic tracking generates a defined EM field in which EM micro sensors are tracked. 6 degree-of-freedom tracking information (spatial position and orientation) may be acquired, for example, by embedding micro sensors into rigid or flexible instruments, where they serve as localization points for the instrument in space. The micro sensors can be embedded, for example, in a coil in the catheter tip. This allows for tracking the catheter tip position inside an electromagnetic field. The EM field generator emits a low intensity, varying EM field that establishes a measurement volume. Small currents are induced inside the sensors when they enter the EM field. The currents are relayed to the sensor interface unit where they are amplified and digitized as signals. The signals are transmitted to the console which calculates each sensor's position and orientation as a transformation.

In some embodiments EM tracking of interventional tools may be also incorporated. Thus, in some embodiments, the 3D position data comprises a spatial position and an orientation of a catheter and/or an interventional tool.

In some embodiments, the 3D pose data may be obtained through optical fiber shape sensing. For example, a low reflectance strain sensor may be positioned in a multi-core fiber within the catheter to determine how a point along the fiber is positioned in space. By sensing the relative change of the sensors in each of three or more fiber cores, the three-dimensional position can be determined.

### Reconstruction of a Patient-Specific 3D Digital Anatomical Model

To generate the interactive patient-specific 3D digital anatomical model, the received data may be processed and combined. Systems of the invention combine multiple image data into a large anatomical representation. The digital anatomical model may be generated in real-time for anatomy that is within the field-of-view.

For example, to generate a patient-specific 3D digital anatomical model based on the 4D image data acquired along the ICE catheter's trajectory over time, as well as to navigate the ICE catheter, the systems of the method utilize medical image registration, computer vision (CV)-based approaches, and/or simultaneous localization and mapping (SLAM). As disclosed in more detail herein, the methods of combination and reconstruction may be, for example, image-based registration, (SLAM), and other computer vision (CV)-based approaches.

Image registration is the process of aligning multiple data, i.e. images, volumes, or surfaces to the patient coordinate system. Systems of the invention utilize one or more registration algorithms to, for example, combine images of the patient and/or data from different modalities and to align temporal sequences of images to generate the interactive and/or real-time patient-specific digital anatomical model. The one or more algorithms find an optimal spatial transformation that best aligns the underlying anatomical structures for reconstruction of the digital anatomical model. Thus, the systems of the invention may use image registration techniques to combine multiple image data into an anatomical representation that combines the multiple image data into a large anatomical representation.

Simultaneous localization and mapping (SLAM) is a computational method that constructs or updates a map of an unknown environment while simultaneously keeping track of an agent's location within it. In some embodiments, systems of the invention adapt and utilize one or more algorithms for SLAM applications to ultrasound image data acquired to generate the patient-specific anatomical model as well as to navigate the catheter. For example, SLAM processing techniques may be adapted for use in conjunction with the catheter utilizing various sensors to incrementally build the map of a patient anatomical environment and simultaneously determine the location of the catheter within the map. Thus, the systems of the invention may use adapted SLAM techniques to combine multiple image data into an anatomical representation that combines the multiple image data into a large anatomical representation.

CV-based image reconstruction approaches utilized by systems of the invention may include approaches based on deep neural networks (DNNs) such as autoencoders (AEs), convolutional neural networks (CNNs), and generative adversarial networks (GANs). The computer vision approaches utilized by systems of the invention aim to detect, interpret and reconstruct data in a way that mimics the intricacy of the human visual system thus providing for intuitive navigation to precisely target an anatomical region.

Further, systems of the invention may adapt and utilize one or more image segmentation algorithms for generating the patient-specific digital anatomical model. Image segmentation is the process of dividing an image into multiple meaningful and homogeneous regions or objects based on their inherent characteristics, such as color, texture, shape, or brightness. In some embodiments, each pixel is labeled, and all pixels belonging to the same category have a common label assigned to them. Segmentation may be achieved via one or more algorithms. For example, in some embodiments, segmentation is achieved via instance image segmentation in which each object in an image is detected and segmented, via one or more algorithms to separate overlapping objects. In some embodiments, segmentation may be achieved via semantic segmentation in which one or more algorithms are used to label each pixel. In some embodiments, segmentation may be achieved via panoptic segmentation in which one or more machine learning algorithms are used to label each pixel with a class label and identifies each object instance in the image to provide for detection and interaction of the object within the environment. Thus, the systems of the invention may use CV-based techniques to combine multiple image data into an anatomical representation that combines the multiple image data into a large anatomical representation.

In some embodiments, the segmentation may be performed by a vision algorithm such as thresholding, connected component analysis, or a neural network based segmentation. In some embodiments, segmentation of the full panoramic volumes may be performed, for example, via a deep learning algorithm.

### Initialization and Continuous 3D Image Registration

In some embodiments, receiving, processing, and combining the received data to reconstruct the interactive digital model of an imaged anatomy includes initialization, continuous 3D image registration, segmentation, filtering, optimization, 3D fusion, and panoramic image reconstruction.

In some embodiments, combining the received data comprises selecting a 3D ultrasound image in the catheter-based ultrasound imaging data and mapping the 3D ultrasound image to the physical patient coordinate system using, for example, EM tracking data. In this way, a registration of each 3D ultrasound image in the physical patient's coordinate system may be initialized. The ultrasound image selected for initialization may be the first or any subsequent ultrasound image. 3D pose data may be used for initialization of the registration. In some embodiments, EM tracking data may be used for initialization of the registration.

As disclosed herein, systems of the invention provide for multimodal image registration using one or more algorithms to correlate morphologic and/or functional features between images. The registration volume may be the entire imaged volume or only a subset of the available image set. A suitable spatial overlap may be necessary to enable registration. Utilizing a rigid registration scheme is beneficial, while a deformable registration scheme can cope with deformations caused by inaccurate ECG filtering and breathing effects. A deformable image registration takes into account non-linear deformation and displacement, and may include geometrically deforming one image into another by applying one or more algorithms, for example deep learning-based models. For example, the deformable registration scheme may be a computational process in which an image similarity measure function and a transformation model are defined for the images of interest, then an optimization algorithm may be used to adjust the transformation model in a way that maximizes the similarity function. The transformation models may include spline and demons, elastic, fluid, finite element model, and free form deformations. Rigid registration is a global match between image sets that preserves the relative distance between every pair of points from the patient's anatomy. Rigid-body registration may include a combination of rotation and translation in order to bring the images into the same coordinate system.

3D ultrasound images, subsequent to the initialization, along the ICE catheter's trajectory may be filtered using pulse phase-gating. For example, the pulse-phase gating may be ECG-gating in some embodiments. 3D ultrasound images of the same cardiac phase may be registered against the respective previous 3D ultrasound image or the current fused 3D image.

Subsequent optimization steps increase the accuracy of the localization of each image in the patient's coordinate system. The optimization steps also ensure robustness of the system. For example, wherein 3D pose data comprises EM tracking data, optimization ensures robustness of the systems against missing EM tracking data, for instance where the ICE catheter leaves the EM tracking field. To provide for improved robustness of registration in the case of inaccurate EM data used for initialization, or in the case of limited image features (e.g. when the ICE catheter is in the inferior vena cava (IVC) next to the lung), an additional tracking scheme may be used. The tracking scheme may use the EM tracking data and the previous registration results to predict the next 3D position. The predicted next 3D position may then be used for initialization of the continuous registration. To further provide for improved registration in regions with few features, prior knowledge of the imaged anatomy may be applied.

In some embodiments, combining the received data further comprises continuously registering subsequent 3D ultrasound images within the physical patient's coordinate system. In some embodiments, the 3D pose data may be combined with one or more previous registration results to predict a next 3D position. For example, where EM tracking data is used, the EM tracking data may be combined with one or more previous registration results to predict a next 3D position.

In some embodiments, the predicted next 3D position may be used for initialization of the continuous registration. As disclosed in more detail herein, in some embodiments, the continuously registered subsequent 3D ultrasound images are partly overlapping such that the continuously registered and partly overlapping subsequent 3D ultrasound images are fused into one panoramic digital model.

The continuous 3D ultrasound image registration may include filtering, using pulse separate digital model may be generated for each cardiac phase. Thus, the 3D ultrasound images of a same cardiac phase may be registered against a respective previous 3D ultrasound image and/or a fused 3D image. Further, in some embodiments, the filtered subsequent 3D ultrasound images are segmented using a supervised or unsupervised deep-learning algorithm.

### Generation of Patient-Specific 3D Digital Anatomical Model

As disclosed herein, the continuously registered subsequent 3D ultrasound images may be partly overlapping such that the continuously registered and partly overlapping subsequent 3D ultrasound images are fused into a large 3D image representing a patient-specific panoramic reconstruction of the anatomy. This 3D panoramic reconstruction may be updated sequentially over time as the catheter is moved through the anatomical region or regions of interest, such that the patient-specific digital 3D anatomical model is continuously updated with subsequent registration and segmentation results.

In some embodiments, the digital anatomical model encapsulates a representation of the anatomy in a spatial topology for live visualization of one or more anatomical regions of interest. The spatial topology comprises a topological map, such that each point in the topological map represents tissue and/or one or more specific anatomical properties. Thus, ultrasound imaging data from one or more directions and/or one or more views may be combined to provide the representation of the anatomy. This representation may be a panoramic image reconstruction (i.e. an intensity volume), a (segmented) surface model, and/or a mesh. The digital anatomy may also be a more advanced representation of the anatomy, where for each point in the topological map, a representation of tissue or specific anatomical properties are encapsulated, such that different views or information from different directions can be combined for a complete representation. Thus, by reconstructing an interactive digital anatomical model, based on the processing and combining of the received data, the systems of the invention provide a digital anatomical model similar to computed tomography (CT), magnetic resonance imaging (MRI), or ultrasound tomographic reconstructions. The digital model encapsulates a representation of the anatomy in a spatial topology for live visualization of one or more anatomical regions of interest.

The digital model may be at least one of a panoramic image reconstruction, a segmented surface model, and a mesh. In some embodiments, the panoramic digital model may be segmented using a deep learning algorithm.

As disclosed herein, the continuous 3D ultrasound image registration may include filtering, using pulse phase-gating, the subsequent 3D ultrasound images along the catheter's trajectory, such that a separate digital model may be generated for each cardiac phase. Thus, the 3D ultrasound images of a same cardiac phase may be registered against a respective previous 3D ultrasound image and/or a fused 3D image. Further, in some embodiments, the filtered subsequent 3D ultrasound images are segmented using a supervised or unsupervised deep-learning algorithm.

Systems of the invention provide for 3D segmentation of the ultrasound imaging data. In some embodiments, relevant structures of the cardiac and vascular anatomy are segmented in ECG-gated 3D ultrasound images. For example, a main focus may be the segmentation of the inner shell (surface) of the right atrium (RA), left atrium (LA), and the traversed veins. The inner shell may be defined as the tissue-blood interface of the blood pool and the cardiac or vascular wall. Because segmentation of ICE data is challenging, systems of the invention include using supervised or unsupervised machine-learning approaches in some embodiments. In some embodiments, the filtered subsequent 3D ultrasound images are segmented using a supervised or unsupervised deep-learning algorithm.

The systems of the invention provide for adaptive 3D model generation. By fusing the segmented 3D shells using the 3D registration results, a patient-specific digital 3D anatomical model may be obtained. The 3D model may be continuously updated with the newest registration and segmentation results.

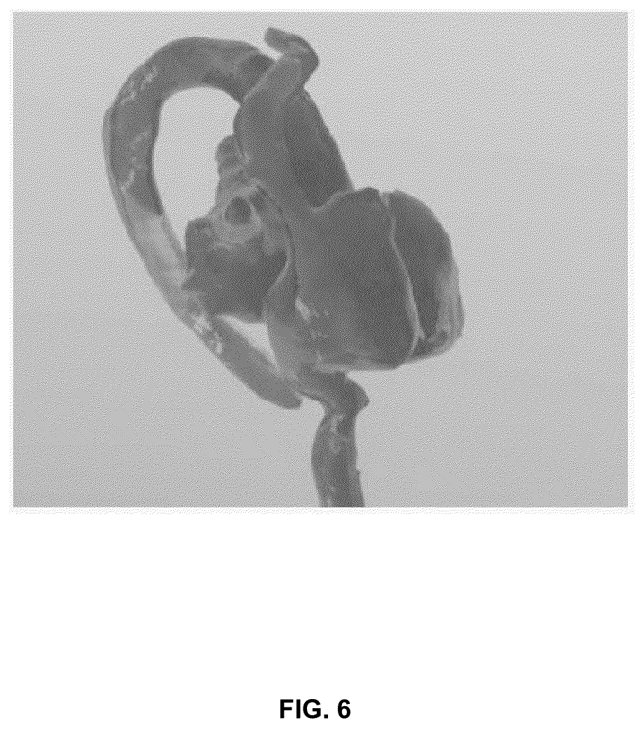

The systems of the invention provide for generation of a 4D dynamic (spatio-temporal) digital model. FIG. 6 illustrates a 4D heart model that may be composed using systems of the invention. The 4D model may be achieved by registration and fusion of ultrasound volumes as described herein. By applying the steps for generating a patient-specific 3D digital anatomical model for different cardiac phases, a separate 3D model may be generated for each cardiac phase. These separate models may be combined into a temporal dynamic model that represents the patient-specific anatomy over time. In some embodiments the temporal dynamic model visualizes the heartbeat as a sequence of animated 3D models. For example, as shown in FIG. 6, the venous access and the heart are visible as a 3D model, reconstructed from the ultrasound image data acquired during the minimally invasive, catheter-based intervention with the ICE catheter. The 3D model may be expanded piece by piece along the trajectory of the ICE catheter in the vessels of the heart. The 4D view visualizes the heartbeat as a sequence of animated models.

In some embodiments, the continuously updated patient-specific digital 3D anatomical model may be visualized simultaneously with the fused ultrasound image data to enable 3D navigation of the catheter in real-time. The systems of the invention provide for generating 3D anatomical landmarks. To aid the navigation of the ICE catheter and interventional tools, a detection and localization step may be added to automatically find prominent anatomical landmarks. These landmarks may be visualized in 3D along with the model.

Systems of the invention provide for tracking of interventional tools within the 3D model. The console may be further configured to detect, localize, and segment in the 3D ultrasound images, relevant interventional tools. For initialization of the detection step, 3D pose data, such as EM tracking data, may be used as available. The segmented tools may be visualized along with the 3D digital anatomical model. In addition to physical tracking and navigation, the invention provides for contact assessment between the tool tip and cardiac or vascular wall.

In some embodiments, the model may be generated once when all of the 3D ultrasound image data is available. After model generation, navigation of the ICE catheter as well as tracking and navigation of interventional tools is possible. Thus, the anatomical context generated with the model enables safer navigation of catheter tools, for example during septal crossing procedures. In some embodiments, the console may be further operable to automatically detect and localize anatomical landmarks for visualization within the digital model to aid navigation of the catheter and interventional tools.

As disclosed herein, in some embodiments, the interactive digital model may be generated beginning from a catheter access site to one or more targets within a heart to provide an interactive visualization of patient-specific structure and/or morphology of intra-cardiac structures without the need for fluoroscopy. For example, the intra-cardiac structures may be one or more of trabeculae, tendons, chordae, and valves/leaflets.

### Methods for Real-Time Reconstruction of a Patient-Specific Anatomical Model

Aspects of the invention provide methods for interactive reconstruction of a digital anatomical model.

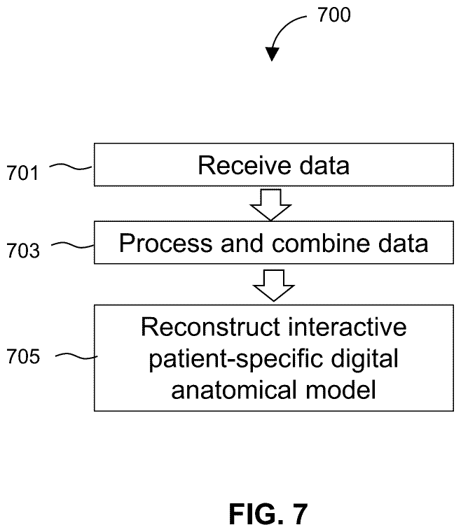

FIG. 7 illustrates a block diagram of a method 700 for interactive reconstruction of a digital anatomical model according to one embodiment. The method includes the steps of receiving 701, via a console operably associated with an ultrasound imaging device, data associated with at least one of cardiac and vascular anatomy, the data comprising catheter-based ultrasound imaging data, and pulse phase data. The method further includes processing and combining 703, via the console, the received data; and reconstructing 705, based on said processing and combining of data, an interactive digital model of an imaged anatomy, such that the digital model encapsulates a representation of the anatomy in a spatial topology for live visualization of one or more anatomical regions of interest.

The console may be operable to process and combine the received data, and reconstruct, based on the processing and combining of data, an interactive digital model of an imaged anatomy. The digital model encapsulates a representation of the anatomy in a spatial topology for live visualization of one or more anatomical regions of interest.

As disclosed herein, some embodiments of the method may include a console in active communication with a computing system configured to communicate across a network. The console may be configured to be operably associated with an ultrasound imaging device, as well as with other devices and/or means for receiving data, and to exchange data therewith. The console may comprise a hardware processor coupled to non-transitory, computer-readable memory containing instructions executable by the processor to cause the console to receive data associated with at least one of cardiac and vascular anatomy the data comprising catheter-based ultrasound imaging data, and, in some embodiments, pulse phase data and/or 3D position data. The console may be operable to process and combine the received data, and reconstruct, based on said processing and combining of data, an interactive digital model of an imaged anatomy, wherein the digital model encapsulates a representation of the anatomy in a spatial topology for live visualization of one or more anatomical regions of interest.

The computing system or computing device may include one or more processors and memory, as well as an input/output mechanism (i.e., a keyboard, knobs, scroll wheels, or the like) with which a user can interact so as to operate the console, including making adjustments to the ultrasound imaging system, saving images, initialization, continuous 3D image registration, filtering, optimization, 3D fusion, and panoramic image reconstruction.

The computing system may include a computer program comprising one or more algorithms for image registration, segmentation, filtering, optimization, and reconstruction of the digital anatomical model. For example, the algorithm may be part of a computer program executable by the computing system and in communication with the console of the system. The systems may be in communication with the imaging device to receive 3D ultrasound image data from the imaging device.

As disclosed in detail herein, imaging protocols and algorithms may be used to dynamically reconstruct properties of the anatomy. The method may include utilizing one or more algorithms for dynamically reconstructing multiple images from the 3D image data to provide a 3D visualization of the anatomical region of interest and targeted tissue site.

### Received Data

The methods of the invention include receiving, processing, and combining real-time catheter-based ultrasound imaging data with other data such as pulse-phase data and/or 3D pose data (position and orientation data) and reconstructing a representation of the digital anatomy based on the receiving, processing, and combining steps. Methods of the invention include generating a reliable and anatomically correct patient-specific 3D digital anatomical model.

### Ultrasound Imaging Data:

Methods of the invention include receiving and processing ultrasound imaging data. In some embodiments of the method, the ultrasound imaging data may be intracardiac ultrasound image data. As such, the method may include using available 3D ICE catheters for acquiring ultra-fast 4D ultrasound image data of a considered cardiac and/or vascular anatomy for processing and combining with other data to generate the reconstructed digital anatomical model.

In some embodiments, methods of the invention utilize multiple fields-of-view, e.g. cylindrical fields-of-view acquired with a rotating transducer array or a cylindrical folded transducer matrix. Thus, cylindrical/360-degree imaging may be applied to provide an extended field-of view (FOV).

3D ICE catheters with forward and with sideways transducer arrays are suitable for use with methods of the invention as long as the field-of-view is sufficiently large, to provide for panoramic reconstruction. For example, catheters facing sideways may be used as these catheters also cover the forward direction to some degree due to the opening angle of the ultrasound beam. Any field-of view implementations may be used to achieve imaging of the whole vascular geometry and its surroundings for each volume such that imaging may be 360 degrees around the catheter. In some embodiments of the methods, the ultrasound imaging data may be received from a plurality of cylindrical fields-of-view. For example, in some embodiments of the methods, the plurality of cylindrical fields of view are acquired via a rotating transducer array and/or a cylindrical folded transducer matrix.

Generally, a whole considered anatomical region is not covered by a single 3D ultrasound image. Therefore, in some embodiments, methods of the invention provide for selecting an appropriate trajectory of the ICE catheter such that the whole region is covered over time by multiple partly overlapping 3D images. Methods of the invention may provide a trajectory starting from the catheter access site up to targets of interest, for example targets within the heart, which enables procedures requiring no fluoroscopy. Methods of the invention provide for reconstructing a digital anatomy from different acquisitions. A clinically important protocol is a trajectory starting from the catheter access site up to the targets within the heart, which enables procedures requiring no fluoroscopy. This is particularly useful in the reconstruction of the intracardiac digital anatomy. The method includes acquiring data during the advancement of the catheter from the access site up into the right atrium. Further data may be acquired during pull back (retraction) from the Superior Vena Cava into the Inferior Vena Cava for reconstruction of a right atrial map and left atrial map. Data may be collected from a pullback from the left-sided pulmonary veins into the septal wall for reconstructing a high resolution left atrial map.

To increase the field-of-view, the catheter's motion may be perturbed slightly from a smooth trajectory in a defined way. For example, the catheter tip may be disturbed or “wobbled” using a built-in steering mechanism, which changes the viewing angle. By doing so, the image contrast of interfaces that are not well aligned with the ultrasound wave front in the smooth trajectory may be improved. Thus, in some embodiments of the methods, the ultrasound imaging data may be received from a field of view whereby a motion of the catheter is perturbed from a smooth trajectory to change the viewing angle. Further, in some embodiments of the method, the speed of the ICE catheter's trajectory may be limited by the field-of-view of the ultrasound image data such that subsequent phase-gated, e.g. ECG-gated, 3D images have sufficient overlap.

### Pulse Phase Data:

Information on cardiac pulse phase may also be received, processed, and combined with the ultrasound imaging data to generate the reconstructed digital anatomical model. Pulse phase data may be received from other systems for pulse phase information such as pulse oximetry, intracardiac activity data from one or more catheters, or image-based methods. In some embodiments of the method, the pulse phase data may be one or more of electrocardiogra (ECG) data, pulse oximetry data, and/or image-based intracardiac activity data. For example, pulse phase data may be received via ECG. The pulse-phase data, such as ECG data, may be used to filter the cardiac phases.

### 3D Position and Orientation Data:

Methods of the invention provide for the real-time 3D localization and tracking of the catheter, for example an ICE catheter, and interventional tools within the patient coordinate system. Methods of the invention may receive 3D position and orientation data of the catheter, and process and combine this data with the ultrasound imaging data and/or the pulse phase data for reconstruction of the digital anatomical model. It is noted however, that in some embodiments of the method, reconstruction of the digital anatomical model is possible without 3D position data. The 3D position and orientation data may be referred to as 3D pose data. 3D pose data may be 6 degree-of-freedom tracking including position and orientation. The 3D pose data may comprise a position and orientation in 3D space.

Tracking data may be used by the console to map the ultrasound image data to a patient coordinate system.

The 3D pose data may be obtained through electromagnetic (EM) tracking, impedance tracking, image-based tracking, fiber-optic shape sensing, and/or a combination of these modalities. In some embodiments of the methods, EM tracking data may be used. Electromagnetic tracking generates a defined EM field in which EM micro sensors are tracked. 6 degree-of-freedom tracking information (spatial position and orientation) may be acquired, for example, by embedding micro sensors into rigid or flexible instruments, where they serve as localization points for the instrument in space. The micro sensors can be embedded, for example, in a coil in the catheter tip. This allows for tracking the catheter tip position inside an electromagnetic field. The EM field generator emits a low intensity, varying EM field that establishes a measurement volume. Small currents are induced inside the sensors when they enter the EM field. The currents are relayed to the sensor interface unit where they are amplified and digitized as signals. The signals are transmitted to the console which calculates each sensor's position and orientation as a transformation.

In some embodiments of the method, EM tracking of interventional tools may be also incorporated. Thus, in some embodiments of the method, the 3D position data comprises a spatial position and an orientation of a catheter and/or an interventional tool.

In some embodiments of the method, the 3D pose data may be obtained through optical fiber shape sensing. For example, low reflectance strain sensors may be positioned in a multi-core fiber within the catheter to determine how a point along the fiber is positioned in space. By sensing the relative change of the sensors in each of three or more fiber cores, the three-dimensional position can be determined.

### Reconstruction of a Patient-Specific 3D Digital Anatomical Model

To generate the interactive patient-specific 3D digital anatomical model, the received data may be processed and combined. Methods of the invention combine multiple image data into a large anatomical representation. The digital anatomical model may be generated in real-time for anatomy that is within the field-of-view.

For example, to generate a patient-specific 3D digital anatomical model based on the 4D image data acquired along the ICE catheter's trajectory over time, as well as to navigate the ICE catheter, the systems of the method utilize medical image registration, computer vision (CV)-based approaches, and/or simultaneous localization and mapping (SLAM). As disclosed in more detail herein, the methods of combination and reconstruction may be, for example, image-based registration, (SLAM), and other computer vision (CV)-based approaches.

Image registration is the process of aligning multiple data, i.e. images, volumes, or surfaces to the patient coordinate system. Methods of the invention utilize one or more registration algorithms to, for example, combine images of the patient and/or data from different modalities and to align temporal sequences of images to generate the interactive and/or real-time patient-specific digital anatomical model. The one or more algorithms find an optimal spatial transformation that best aligns the underlying anatomical structures for reconstruction of the digital anatomical model. Thus, the methods of the invention may use image registration techniques to combine multiple image data into an anatomical representation that combines the multiple image data into a large anatomical representation.

Simultaneous localization and mapping (SLAM) is a computational method that constructs or updates a map of an unknown environment while simultaneously keeping track of an agent's location within it. In some embodiments, methods of the invention adapt and utilize one or more algorithms for SLAM applications to ultrasound image data acquired to generate the patient-specific anatomical model as well as to navigate the catheter. For example, SLAM processing techniques may be adapted for use in conjunction with the catheter utilizing various sensors to incrementally build the map of a patient anatomical environment and simultaneously determine the location of the catheter within the map. Thus, the methods of the invention may use adapted SLAM techniques to combine multiple image data into an anatomical representation that combines the multiple image data into a large anatomical representation.

CV-based image reconstruction approaches utilized by methods of the invention may include approaches based on deep neural networks (DNNs) such as autoencoders (AEs), convolutional neural networks (CNNs), and generative adversarial networks (GANs). The computer vision approaches utilized by methods of the invention aim to detect, interpret and reconstruct data in a way that mimics the intricacy of the human visual system thus providing for intuitive navigation to precisely target an anatomical region.

Further, methods of the invention may adapt and utilize one or more image segmentation algorithms for generating the patient-specific digital anatomical model. Image segmentation is the process of dividing an image into multiple meaningful and homogeneous regions or objects based on their inherent characteristics, such as color, texture, shape, or brightness. In some embodiments of the methods, each pixel is labeled, and all pixels belonging to the same category have a common label assigned to them. Segmentation may be achieved via one or more algorithms. For example, in some embodiments of the methods, segmentation is achieved via instance image segmentation in which each object in an image is detected and segmented, via one or more algorithms to separate overlapping objects. In some embodiments of the methods, segmentation may be achieved via semantic segmentation in which one or more algorithms are used to label each pixel. In some embodiments of the methods, segmentation is achieved via panoptic segmentation in which one or more machine learning algorithms are used to label each pixel with a class label and identifies each object instance in the image to provide for detection and interaction of the object within the environment. Thus, the methods of the present invention may use CV-based techniques to combine multiple image data into an anatomical representation that combines the multiple image data into a large anatomical representation. In some embodiments, the segmentation may be performed by a vision algorithm such as thresholding, connected component analysis, or a neural network based segmentation. In some embodiments, segmentation of the full panoramic volumes may be performed, for example, via a deep learning algorithm.

### Initialization and Continuous 3D Image Registration

In some embodiments of the methods, receiving, processing, and combining the received data to reconstruct the interactive digital model of an imaged anatomy includes initialization, continuous 3D image registration, filtering, segmentation, optimization, 3D fusion, and panoramic image reconstruction.

In some embodiments of the methods, combining the received data comprises selecting a 3D ultrasound image in the catheter-based ultrasound imaging data and mapping the 3D ultrasound image to the physical patient coordinate system using, for example, EM tracking data. In this way, a registration of each 3D ultrasound image in the physical patient's coordinate system may be initialized. The ultrasound image selected for initialization may be the first or any subsequent ultrasound image. 3D pose data may be used for initialization of the registration. In some embodiments of the methods, EM tracking data may be used for initialization of the registration.

As disclosed herein, methods of the invention provide for multimodal image registration using one or more algorithms to correlate morphologic and/or functional features between images. The registration volume may be the entire imaged volume or only a subset of the available image set. A suitable spatial overlap may be necessary to enable registration. Utilizing a rigid registration scheme may be beneficial, while a deformable registration scheme can cope with deformations caused by inaccurate ECG filtering and breathing effects. A deformable image registration takes into account non-linear deformation and displacement, and may include geometrically deforming one image into another by applying one or more algorithms, for example deep learning-based models. For example, the deformable registration scheme may be a computational process in which an image similarity measure function and a transformation model are defined for the images of interest, then an optimization algorithm may be used to adjust the transformation model in a way that maximizes the similarity function. The transformation models may include spline and demons, elastic, fluid, finite element model, and free form deformations. Rigid registration is a global match between image sets that preserves the relative distance between every pair of points from the patient's anatomy. Rigid-body registration may include a combination of rotation and translation in order to bring the images into the same coordinate system.

3D ultrasound images, subsequent to the initialization, along the ICE catheter's trajectory may be filtered using pulse phase-gating. For example, the pulse-phase gating may be ECG-gating in some embodiments. 3D ultrasound images of the same cardiac phase may be registered against the respective previous 3D ultrasound image or the current fused 3D image.

Subsequent optimization steps increase the accuracy of the localization of each image in the patient's coordinate system. The optimization steps also ensure robustness of the system. For example, wherein 3D pose data comprises EM tracking data, optimization ensures robustness of the systems against missing EM tracking data, for instance where the ICE catheter leaves the EM tracking field. To provide for improved robustness of registration in the case of inaccurate EM data used for initialization, or in the case of limited image features (e.g. when the ICE catheter is in the inferior vena cava (IVC) next to the lung), an additional tracking scheme may be used. The tracking scheme may use the EM tracking data and the previous registration results to predict the next 3D position. The predicted next 3D position may then be used for initialization of the continuous registration. To further provide for improved registration in regions with few features, prior knowledge of the imaged anatomy may be applied.

In some embodiments of the method, combining the received data further comprises continuously registering subsequent 3D ultrasound images within the physical patient's coordinate system. In some embodiments of the method, the 3D pose data may be combined with one or more previous registration results to predict a next 3D position. For example, where EM tracking data is used, the EM tracking data may be combined with one or more previous registration results to predict a next 3D position.

In some embodiments of the method, the predicted next 3D position may be used for initialization of the continuous registration. As disclosed in more detail herein, in some embodiments of the method, the continuously registered subsequent 3D ultrasound images are partly overlapping such that the continuously registered and partly overlapping subsequent 3D ultrasound images are fused into one panoramic digital model.

The continuous 3D ultrasound image registration may include filtering, using pulse separate digital model may be generated for each cardiac phase. Thus, the 3D ultrasound images of a same cardiac phase may be registered against a respective previous 3D ultrasound image and/or a fused 3D image. Further, in some embodiments, the filtered subsequent 3D ultrasound images are segmented using a supervised or unsupervised deep-learning algorithm.

### Generation of Patient-Specific 3D Digital Anatomical Model

As disclosed herein, the continuously registered subsequent 3D ultrasound images may be partly overlapping such that the continuously registered and partly overlapping subsequent 3D ultrasound images are fused into a large 3D image representing a patient-specific panoramic reconstruction of the anatomy. This 3D panoramic reconstruction may be updated sequentially over time as the catheter is moved through the anatomical region or regions of interest, such that the patient-specific digital 3D anatomical model may be continuously updated with subsequent registration and segmentation results.

In some embodiments of the method, the digital anatomical model encapsulates a representation of the anatomy in a spatial topology for live visualization of one or more anatomical regions of interest. The spatial topology comprises a topological map, such that each point in the topological map represents tissue and/or one or more specific anatomical properties. Thus, ultrasound imaging data from one or more directions and/or one or more views may be combined to provide the representation of the anatomy. This representation may be a panoramic image reconstruction (i.e. an intensity volume), a (segmented) surface model, and/or a mesh. The digital anatomy may also be a more advanced representation of the anatomy, where for each point in the topological map, a representation of tissue or specific anatomical properties are encapsulated, such that different views or information from different directions can be combined for a complete representation. Thus, by reconstructing an interactive digital anatomical model, based on the processing and combining of the received data, the systems of the invention provide a digital anatomical model similar to computed tomography (CT), magnetic resonance imaging (MRI), or ultrasound tomographic reconstructions. The digital model encapsulates a representation of the anatomy in a spatial topology for live visualization of one or more anatomical regions of interest.

The digital model may be at least one of a panoramic image reconstruction, a segmented surface model, and a mesh. In some embodiments of the methods, the panoramic digital model may be segmented using a deep learning algorithm.

As disclosed herein, the continuous 3D ultrasound image registration may include filtering, using pulse phase-gating, the subsequent 3D ultrasound images along the catheter's trajectory, such that a separate digital model may be generated for each cardiac phase. Thus, the 3D ultrasound images of a same cardiac phase may be registered against a respective previous 3D ultrasound image and/or a fused 3D image. Further, in some embodiments of the methods, the filtered subsequent 3D ultrasound images are segmented using a supervised or unsupervised deep-learning algorithm.

Methods of the invention provide for 3D segmentation of the ultrasound imaging data. In some embodiments, relevant structures of the cardiac and vascular anatomy are segmented in ECG-gated 3D ultrasound images. For example, a main focus may be the segmentation of the inner shell (surface) of the right atrium (RA), left atrium (LA), and the traversed veins. The inner shell may be defined as the tissue-blood interface of the blood pool and the cardiac or vascular wall. Because segmentation of ICE data is challenging, systems of the invention include using supervised and/or unsupervised machine-learning approaches in some embodiments. In some embodiments of the methods, the filtered subsequent 3D ultrasound images are segmented using a supervised or unsupervised deep-learning algorithm.

The methods of the invention provide for adaptive 3D model generation. By fusing the segmented 3D shells using the 3D registration results, a patient-specific digital 3D anatomical model may be obtained. The 3D model may be continuously updated with the newest registration and segmentation results.

The methods of the invention provide for generation of a 4D dynamic (spatio-temporal) digital model. The 4D model may be achieved by registration and fusion of ultrasound volumes as described herein. By applying the steps for generating a patient-specific 3D digital anatomical model for different cardiac phases, a separate 3D model may be generated for each cardiac phase. These separate models may be combined into a temporal dynamic model that represents the patient-specific anatomy over time. In some embodiments of the methods, the temporal dynamic model visualizes the heartbeat as a sequence of animated 3D models. The 3D model may be expanded piece by piece along the trajectory of the ICE catheter in the vessels of the heart. The 4D view visualizes the heartbeat as a sequence of animated models.

In some embodiments of the method, the continuously updated patient-specific digital 3D anatomical model may be visualized simultaneously with the fused ultrasound image data to enable 3D navigation of the catheter in real-time. The methods of the invention provide for generating 3D anatomical landmarks. To aid the navigation of the ICE catheter and interventional tools, a detection and localization step may be added to automatically find prominent anatomical landmarks. These landmarks may be visualized in 3D along with the model.

Methods of the invention provide for tracking of interventional tools within the 3D model. The console may be further configured to detect, localize, and segment in the 3D ultrasound images, relevant interventional tools. For initialization of the detection step, 3D pose data, such as EM tracking data, may be used as available. The segmented tools may be visualized along with the 3D digital anatomical model. In addition to physical tracking and navigation, the invention provides for contact assessment between the tool tip and cardiac or vascular wall.

In some embodiments of the method, the model may be generated once when all of the 3D ultrasound image data is available. After model generation, navigation of the ICE catheter as well as tracking and navigation of interventional tools is possible. Thus, the anatomical context generated with the model enables safer navigation of catheter tools, for example during septal crossing procedures.

Systems for Interactive Catheter and/or Interventional Tool Tracking

As disclosed herein, aspects of the present invention provide systems for interactive catheter and/or interventional tool tracking.

The systems include a console configured to be operably associated with an ultrasound imaging device and exchange data therewith, the console comprising a hardware processor coupled to non-transitory, computer-readable memory containing instructions executable by the processor. The instructions executable by the processor cause the console to receive data associated with at least one of cardiac and vascular anatomy, the data comprising catheter-based ultrasound imaging data, and pulse phase data. Further, the console may be operable to process and combine the received data, and reconstruct, based on said processing and combining of data, an interactive digital model of a catheter and/or interventional tool in an imaged anatomy, such that he digital model encapsulates a representation of the catheter and/or interventional tool in a spatial anatomical topology for live visualization of the catheter and/or interventional tool in one or more anatomical regions of interest.

The systems may be configured to detect, localize, and segment one or more interventional tools within the 3D ultrasound images, using the methods disclosed herein. Thus, the segmented interventional tools are visualized in 3D within the interactive digital anatomical model.

In some embodiments, the received data further comprises position data comprising a spatial position and an orientation of the catheter and/or an interventional tool. In some embodiments, the console may be further operable to automatically detect and localize anatomical landmarks for visualization within the digital model to aid navigation of the catheter and/or interventional tool. For example, in some embodiments, the detection, localization, and segmentation of the one or more interventional tools provides for contact assessment between a tool tip and a cardiac and/or a vascular wall.

As disclosed herein the systems receive, process, and combine real-time catheter-based ultrasound imaging data with other data such as pulse-phase data and/or 3D pose data (position and orientation data) to reconstruct a representation of the digital anatomy. Systems of the invention then further utilize 3D position data to detect and localize in real-time, near real-time, and/or interactively, the catheter or interventional tool within the digital model.

In some embodiments, the ultrasound imaging data may be intracardiac ultrasound image data. As such, the systems may use available 3D ICE catheters for acquiring ultra-fast 4D ultrasound image data of a considered cardiac and/or vascular anatomy for processing and combining with other data to generate the reconstructed digital anatomical model. In some embodiments, systems of the invention utilize multiple fields-of-view, e.g. cylindrical fields-of-view acquired with a rotating transducer array or a cylindrical folded transducer matrix. Thus, cylindrical/360-degree imaging may be applied to provide an extended field-of view (FOV). In some embodiments, the ultrasound imaging data may be received from a plurality of cylindrical fields-of-view. For example, in some embodiments, the plurality of cylindrical fields of view are acquired via a rotating transducer array and/or a cylindrical folded transducer matrix. In some embodiments, systems of the invention provide for selecting an appropriate trajectory of the ICE catheter such that the whole region may be covered over time by multiple partly overlapping 3D images. Systems of the invention may provide a trajectory starting from the catheter access site up to targets of interest, for example targets within the heart, which enables procedures requiring no fluoroscopy.

To increase the field-of-view, the catheter's motion may be perturbed slightly from a smooth trajectory in a defined way. For example, the catheter tip may be disturbed or “wobbled” using a built-in steering mechanism, which changes the viewing angle. By doing so, the image contrast of interfaces that are not well aligned with the ultrasound wave front in the smooth trajectory may be improved. Thus, in some embodiments, the ultrasound imaging data may be received from a field of view whereby a motion of the catheter is perturbed from a smooth trajectory to change the viewing angle. Further, in some embodiments, the speed of the ICE catheter's trajectory may be limited by the field-of-view of the ultrasound image data such that subsequent phase-gated, e.g. ECG-gated, 3D images have sufficient overlap.

Information on cardiac pulse phase may also be received, processed, and combined with the ultrasound imaging data to generate the reconstructed digital anatomical model. Pulse phase data may be received from other systems for pulse phase information such as pulse oximetry, intracardiac activity data from one or more catheters, or image-based methods. In some embodiments, the pulse phase data may be one or more of electrocardiogramata, pulse oximetry data, and/or image-based intracardiac activity data. For example, pulse phase data may be received via ECG. The pulse-phase data, such as ECG data, may be used to filter the cardiac phases.

Systems of the invention provide for the real-time 3D localization and tracking of the catheter and/or interventional tool within the patient coordinate system and visualized within the digital anatomical mode. Systems of the invention may receive 3D position and orientation data of the catheter and/or interventional tool, and process and combine this data with the ultrasound imaging data and/or the pulse phase data for reconstruction of the digital anatomical model. As disclosed herein, the 3D position and orientation data may be referred to as 3D pose data. 3D pose data may be 6 degree-of-freedom tracking including position and orientation. The 3D pose data may comprise a position and orientation in 3D space. Tracking data may be used by the console to map the ultrasound image data to a patient coordinate system.

Tracking data of interventional tools and/or the catheter may be incorporated into the digital anatomical model. Thus, in some embodiments, the 3D position data comprises a spatial position and an orientation of a catheter and/or an interventional tool. The 3D pose data may be obtained through electromagnetic (EM) tracking, impedance tracking, image-based tracking, fiber-optic shape sensing, and/or a combination of these modalities. In some embodiments, EM tracking data may be used. Electromagnetic tracking generates a defined EM field in which EM micro sensors are tracked. 6 degree-of-freedom tracking information (spatial position and orientation) may be acquired, for example, by embedding micro sensors into rigid or flexible instruments, where they serve as localization points for the instrument in space. The micro sensors can be embedded, for example, in a coil in the catheter tip. This allows for tracking the catheter tip position inside an electromagnetic field. The EM field generator emits a low intensity, varying EM field that establishes a measurement volume. Small currents are induced inside the sensors when they enter the EM field. The currents are relayed to the sensor interface unit where they are amplified and digitized as signals. The signals are transmitted to the console which calculates each sensor's position and orientation as a transformation.

In some embodiments, the 3D pose data may be obtained through optical fiber shape sensing. For example, low reflectance strain sensors may be positioned in a multi-core fiber within the catheter to determine how a point along the fiber is positioned in space. By sensing the relative change of the sensors in each of three or more fiber cores, the three-dimensional position can be determined.

To generate the interactive patient-specific 3D digital anatomical model, the received data may be processed and combined. Systems of the invention combine multiple image data into a large anatomical representation. The digital anatomical model may be generated in real-time for anatomy that is within the field-of-view. As disclosed herein, to generate a patient-specific 3D digital anatomical model based on the 4D image data acquired along the ICE catheter's trajectory over time, as well as to navigate the ICE catheter, the systems of the method utilize medical image registration, computer vision (CV)-based approaches, and/or simultaneous localization and mapping (SLAM).

As disclosed herein, image registration is the process of aligning multiple data, i.e. images, volumes, or surfaces to the patient coordinate system. Systems of the invention utilize one or more registration algorithms to, for example, combine images of the patient and/or data from different modalities and to align temporal sequences of images to generate the interactive and/or real-time patient-specific digital anatomical model. The one or more algorithms find an optimal spatial transformation that best aligns the underlying anatomical structures for reconstruction of the digital anatomical model. Thus, the systems of the invention may use image registration techniques to combine multiple image data into an anatomical representation that combines the multiple image data into a large anatomical representation.

SLAM is a computational method that constructs or updates a map of an unknown environment while simultaneously keeping track of an agent's location within it. In some embodiments, systems of the invention adapt and utilize one or more algorithms for SLAM applications to ultrasound image data acquired to generate the patient-specific anatomical model as well as to navigate the catheter. For example, SLAM processing techniques may be adapted for use in conjunction with the catheter utilizing various sensors to incrementally build the map of a patient anatomical environment and simultaneously determine the location of the catheter within the map. Thus, the systems of the invention may use adapted SLAM techniques to combine multiple image data into an anatomical representation that combines the multiple image data into a large anatomical representation.

CV-based image reconstruction approaches utilized by systems of the invention may include approaches based on deep neural networks (DNNs) such as autoencoders (AEs), convolutional neural networks (CNNs), and generative adversarial networks (GANs). The computer vision approaches utilized by systems of the invention aim to detect, interpret and reconstruct data in a way that mimics the intricacy of the human visual system thus providing for intuitive navigation to precisely target an anatomical region.

Further, systems of the invention may adapt and utilize one or more image segmentation algorithms for generating the patient-specific digital anatomical model. Image segmentation is the process of dividing an image into multiple meaningful and homogeneous regions or objects based on their inherent characteristics, such as color, texture, shape, or brightness. In some embodiments, each pixel is labeled, and all pixels belonging to the same category have a common label assigned to them. Segmentation may be achieved via one or more algorithms. For example, in some embodiments, segmentation is achieved via instance image segmentation in which each object in an image is detected and segmented, via one or more algorithms to separate overlapping objects. In some embodiments, segmentation may be achieved via semantic segmentation in which one or more algorithms are used to label each pixel. In some embodiments, segmentation is achieved via panoptic segmentation in which one or more machine learning algorithms are used to label each pixel with a class label and identifies each object instance in the image to provide for detection and interaction of the object within the environment. Thus, the systems of the invention may use CV-based techniques to combine multiple image data into an anatomical representation that combines the multiple image data into a large anatomical representation. In some embodiments, segmentation of the full panoramic volumes is performed, for example, via a deep learning algorithm.

In some embodiments, receiving, processing, and combining the received data to reconstruct the interactive digital model of an imaged anatomy includes initialization, continuous 3D image registration, filtering, segmentation, optimization, 3D fusion, and panoramic image reconstruction.

In some embodiments, combining the received data comprises selecting a 3D ultrasound image in the catheter-based ultrasound imaging data and mapping the 3D ultrasound image to the physical patient coordinate system using, for example, EM tracking data. In this way, a registration of each 3D ultrasound image in the physical patient's coordinate system is initialized. The ultrasound image selected for initialization may be the first or any subsequent ultrasound image. 3D pose data may be used for initialization of the registration. In some embodiments, EM tracking data may be used for initialization of the registration.

As disclosed herein, systems of the invention provide for multimodal image registration using one or more algorithms to correlate morphologic and/or functional features between images. The registration volume may be the entire imaged volume or only a subset of the available image set. A suitable spatial overlap may be necessary to enable registration. Utilizing a rigid registration scheme is beneficial, while a deformable registration scheme can cope with deformations caused by inaccurate ECG filtering and breathing effects. A deformable image registration takes into account non-linear deformation and displacement, and may include geometrically deforming one image into another by applying one or more algorithms, for example deep learning-based models. For example, the deformable registration scheme may be a computational process in which an image similarity measure function and a transformation model are defined for the images of interest, then an optimization algorithm is used to adjust the transformation model in a way that maximizes the similarity function. The transformation models may include spline and demons, elastic, fluid, finite element model, and free form deformations. Rigid registration is a global match between image sets that preserves the relative distance between every pair of points from the patient's anatomy. Rigid-body registration may include a combination of rotation and translation in order to bring the images into the same coordinate system.

3D ultrasound images, subsequent to the initialization, along the ICE catheter's trajectory may be filtered using pulse phase-gating. For example, the pulse-phase gating may be ECG-gating in some embodiments. 3D ultrasound images of the same cardiac phase may be registered against the respective previous 3D ultrasound image or the current fused 3D image.

Subsequent optimization steps increase the accuracy of the localization of each image in the patient's coordinate system. The optimization steps also ensure robustness of the system. For example, wherein 3D pose data comprises EM tracking data, optimization ensures robustness of the systems against missing EM tracking data, for instance where the ICE catheter leaves the EM tracking field. To provide for improved robustness of registration in the case of inaccurate EM data used for initialization, or in the case of limited image features (e.g. when the ICE catheter is in the inferior vena cava (IVC) next to the lung), an additional tracking scheme may be used. The tracking scheme may use the EM tracking data and the previous registration results to predict the next 3D position. The predicted next 3D position may then be used for initialization of the continuous registration. To further provide for improved registration in regions with few features, prior knowledge of the imaged anatomy may be applied.

In some embodiments, combining the received data further comprises continuously registering subsequent 3D ultrasound images within the physical patient's coordinate system. In some embodiments, the 3D pose data may be combined with one or more previous registration results to predict a next 3D position. For example, where EM tracking data is used, the EM tracking data may be combined with one or more previous registration results to predict a next 3D position.

In some embodiments, the predicted next 3D position may be used for initialization of the continuous registration. As disclosed in more detail herein, in some embodiments, the continuously registered subsequent 3D ultrasound images are partly overlapping such that the continuously registered and partly overlapping subsequent 3D ultrasound images are fused into one panoramic digital model.

The continuous 3D ultrasound image registration may include filtering, using pulse separate digital model is generated for each cardiac phase. Thus, the 3D ultrasound images of a same cardiac phase may be registered against a respective previous 3D ultrasound image and/or a fused 3D image. Further, in some embodiments, the filtered subsequent 3D ultrasound images are segmented using a supervised or unsupervised deep-learning algorithm.

As disclosed herein, the continuously registered subsequent 3D ultrasound images may be partly overlapping such that the continuously registered and partly overlapping subsequent 3D ultrasound images are fused into a large 3D image representing a patient-specific panoramic reconstruction of the anatomy. This 3D panoramic reconstruction may be updated sequentially over time as the catheter is moved through the anatomical region or regions of interest, such that the patient-specific digital 3D anatomical model is continuously updated with subsequent registration and segmentation results.

Systems of the invention provide for tracking of interventional tools within the 3D model. The console may be further configured to detect, localize, and segment in the 3D ultrasound images, relevant interventional tools. For initialization of the detection step, 3D pose data, such as EM tracking data, may be used as available. The segmented tools may be visualized along with the 3D digital anatomical model. In addition to physical tracking and navigation, the invention provides for contact assessment between the tool tip and cardiac or vascular wall.

In some embodiments, the digital anatomical model encapsulates a representation of the anatomy in a spatial topology for live visualization of one or more anatomical regions of interest. The spatial topology comprises a topological map, such that each point in the topological map represents tissue and/or one or more specific anatomical properties. Thus, ultrasound imaging data from one or more directions and/or one or more views may be combined to provide the representation of the anatomy. This representation may be a panoramic image reconstruction (i.e. an intensity volume), a (segmented) surface model, and/or a mesh. The digital anatomy may also be a more advanced representation of the anatomy, where for each point in the topological map, a representation of tissue or specific anatomical properties are encapsulated, such that different views or information from different directions can be combined for a complete representation. Thus, by reconstructing an interactive digital anatomical model, based on the processing and combining of the received data, the systems of the invention provide a digital anatomical model similar to computed tomography (CT), magnetic resonance imaging (MRI), or ultrasound tomographic reconstructions. The digital model encapsulates a representation of the anatomy in a spatial topology for live visualization of one or more anatomical regions of interest.

The digital model may be at least one of a panoramic image reconstruction, a segmented surface model, and a mesh. In some embodiments, the panoramic digital model is segmented using a deep learning algorithm.

As disclosed herein, the continuous 3D ultrasound image registration may include filtering, using pulse phase-gating, the subsequent 3D ultrasound images along the catheter's trajectory, such that a separate digital model is generated for each cardiac phase. Thus, the 3D ultrasound images of a same cardiac phase may be registered against a respective previous 3D ultrasound image and/or a fused 3D image. Further, in some embodiments, the filtered subsequent 3D ultrasound images are segmented using a supervised or unsupervised deep-learning algorithm. In some embodiments, the filtered subsequent 3D ultrasound images are segmented using a supervised or unsupervised deep-learning algorithm. The systems of the invention provide for adaptive 3D model generation. By fusion of the segmented 3D shells using the 3D registration results, a patient-specific digital 3D anatomical model is obtained. The 3D model is continuously updated with the newest registration and segmentation results such that the catheter and/or interventional tool is visualized within the anatomical model.

The systems of the invention provide for generation of a 4D dynamic (spatio-temporal) digital model. The 4D model is achieved by registration and fusion of ultrasound volumes as described herein. By applying the steps for generating a patient-specific 3D digital anatomical model for different cardiac phases, a separate 3D model is generated for each cardiac phase. These separate models may be combined into a temporal dynamic model that represents the patient-specific anatomy over time. In some embodiments the temporal dynamic model visualizes the heartbeat as a sequence of animated 3D models. The 3D model is expanded piece by piece along the trajectory of the ICE catheter in the vessels of the heart. The 4D view visualizes the heartbeat as a sequence of animated models.

In some embodiments, the continuously updated patient-specific digital 3D anatomical model may be visualized simultaneously with the fused ultrasound image data to enable 3D navigation of the catheter in real-time. The systems of the invention provide for generating 3D anatomical landmarks. To aid the navigation of the ICE catheter and interventional tools, a detection and localization step may be added to automatically find prominent anatomical landmarks. These landmarks may be visualized in 3D along with the model.

In some embodiments, the model is generated once when all of the 3D ultrasound image data is available. After model generation, navigation of the ICE catheter as well as tracking and navigation of interventional tools is possible. Thus, the anatomical context generated with the model enables safer navigation of catheter tools, for example during septal crossing procedures. In some embodiments, the console is further operable to automatically detect and localize anatomical landmarks for visualization within the digital model to aid navigation of the catheter and interventional tools.

As disclosed herein, the systems and methods of the invention provide for panoramic anatomical context in 3D intracardiac echocardiography (ICE) with 3D registration and geometry-based image fusion. The invention recognizes that the unfamiliarity of physicians with ICE and a limited field of view can hinder an efficient use of ICE during cardiac interventions. Thus, the systems and methods of the invention provide additional anatomical context in ICE imaging to helps to reduce the procedure time and radiation dosage to patient and physicians. The systems and methods utilize synchronized 360° ultrasound, ECG and 3D tracking data to create anatomical context.

As disclosed herein, the systems and methods of the invention combine one or more modalities to reconstruct a patient-specific anatomy. In some embodiments strain plus data from one or more other methods, e.g. Nakagami or Rayleigh distribution parameters, may be used. In non-limiting examples, for cardiac interventional procedures such as ablation, envelop statistics data, such as Nakagami, may be used in static scenarios or masked scenarios such as a wall mask, and data may be included to further define a lesion width or depth for heart ablation characterization. Further, data from micro-structural methods, such as elastography, may be included, for example to identify regions without tracking. As disclosed herein, the systems and methods accomplish patient-specific tissue reconstruction by utilizing data related to the extraction of perfusion, stiffness, strain, anisotropy, coherence, specific statistical distributions in tissue (Rayleigh, Nakagami), spectral parameters of tissue (frequency power spectrum) and other parameters. Further, the systems and methods of the invention may utilize analysis methods that incorporate data such as ultrasound data at various beamforming stages, learned features from raw and/or processed signals, deep-learning-based methods incorporating 2D, 3D, and 4D deep learning, compounded, envelope, and/or log-compressed signal beamforming stage, full 4D temporal evaluation for dimensionality, tracking integration, i.e. integrated catheter and breathing tracking, position/width/depth data for lesion output. The captured data may be processed using the disclosed protocols to extract anatomical and functional information, and tissue characteristics.

As illustrated in FIG. 5 and disclosed herein, the systems and methods of the invention provide for improved pullback sequence visualization, i.e. during slowly retracting the imaging catheter from the superior vena cava (SVC) through the right atrium (RA) to the inferior vena cava (IVC) to capture the full cardiac anatomy. The systems and methods of the invention provide for correcting tracking error and breathing motion during a pullback sequence. The systems and methods of the invention recognize that full correction of the breathing motion is likely not possible with rigid registration alone. Thus, in some embodiments, the systems and methods utilize a rigid pairwise registration using tracking data as an initial estimate. The systems and methods may use one or more modalities for preprocessing for registration such as, for example, ECG-gating to select corresponding frames, iMAP2 beamforming3 to minimize the presence of aberrations, reducing speckle pattern using the speckle2speckle filter, and utilizing a similarity metric, i.e. mutual information.

As disclosed herein, the systems and methods of the invention may use one or more data sources and modalities for generating a patient-specific anatomical model. The systems and methods of the invention may use ultrasound imaging modalities at various beam-forming stages, as opposed to just the final B-mode. The systems and methods of the invention may use a measurement type that includes learned features from raw/processed signals. The systems and methods of the invention may include 2D/3D/4D deep learning in providing live visualization of complex anatomy in sufficient detail for intuitive navigation to precise locations within the anatomy. Systems and methods of the invention may use imaging data and various other data combined with image segmentation and registration-based or mapping-based techniques as well as 2D/3D/4D deep learning to provide for the reconstruction of a real-time or near real-time interactive patient-specific digital anatomical model. For example, in some embodiments, the systems and methods of the invention combine custom feature extraction with deep learning for lesion characterization. Systems and methods of the invention may utilize a compounded, envelope, and/or log-compressed signal stage. Systems of the invention may use full temporal evolution for increased dimensionality. The systems of the invention may also integrate catheter and breathing tracking data, and incorporate tracking data in a 4D context. The systems of the invention provide for position, width, and depth for displaying lesion (lesion output). Thus, the systems and methods of the invention provide for outputting quantitative lesion dimensions rather than a simple classification as output.

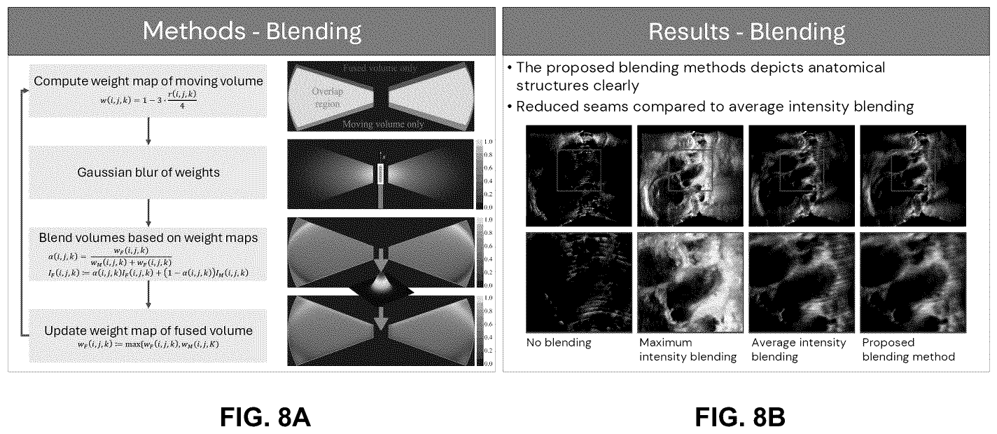

FIG. 8A illustrates a blending method according to one embodiment of the invention. As illustrated, the blending method may include computing a weight map of a moving volume, applying a Gaussian blur of weight, blending the volumes based on weight maps, and updating the weight map of the fused volume.

FIG. 8B illustrates visual results of the blending methods of the invention as compared to average intensity blending. As illustrated, the blending methods of the invention depicts anatomical structures more clearly as compared to traditional methods, and provides reduced seams as compared to average intensity blending.

Thus, the systems and methods of the invention provide for successful reconstruction of patient-specific cardiac anatomy based on registration and fusion of 3D ICE data. The blending strategy, as disclosed herein, depicts anatomical structures in a panoramic ultrasound such that there is a high degree of agreement of the constructed panoramic ultrasound with preoperative CT scans.

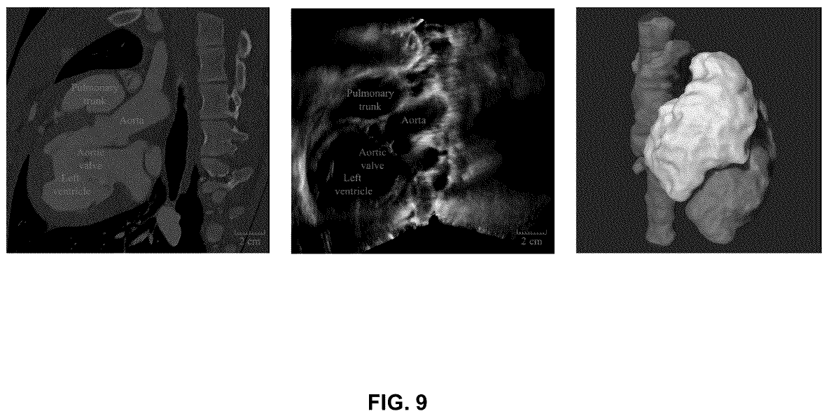

FIG. 9 illustrates a reconstruction of patient specific cardiac anatomy according to one embodiment of the systems and methods of the invention.

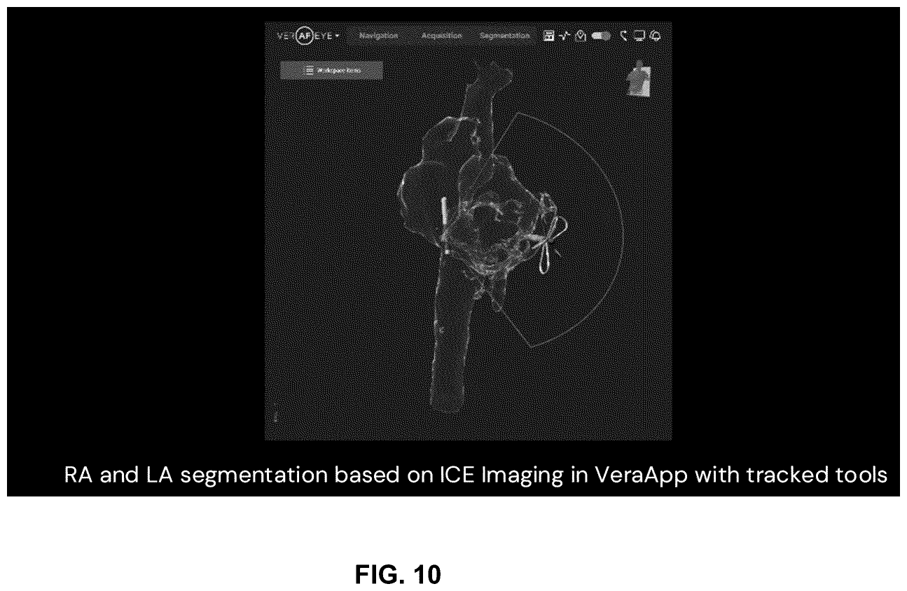

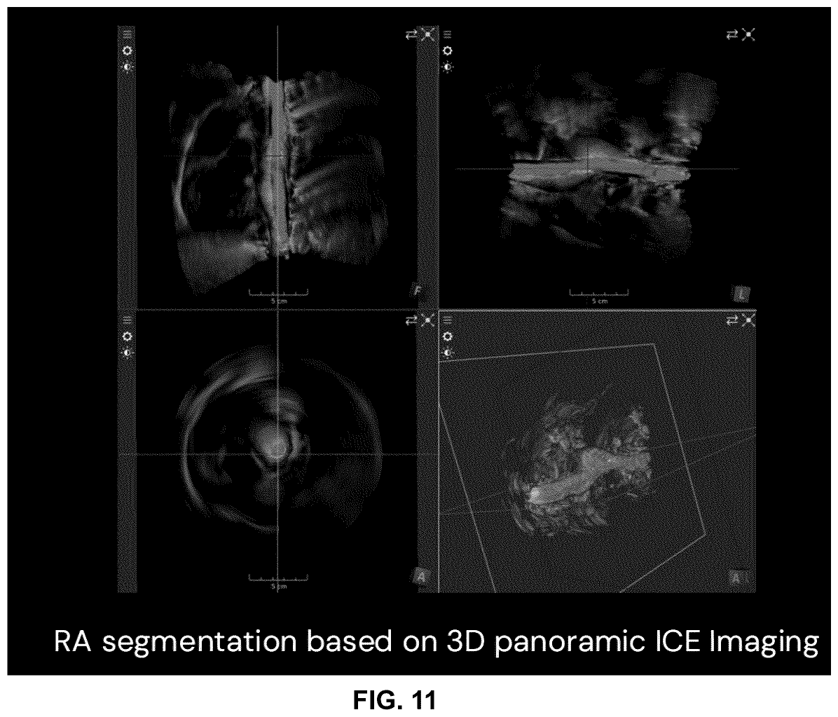

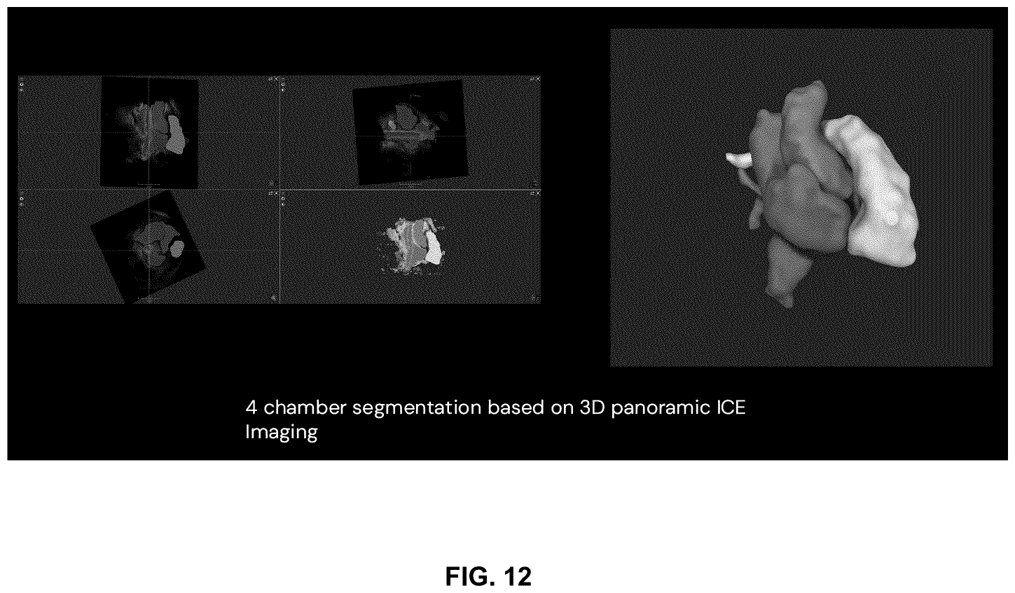

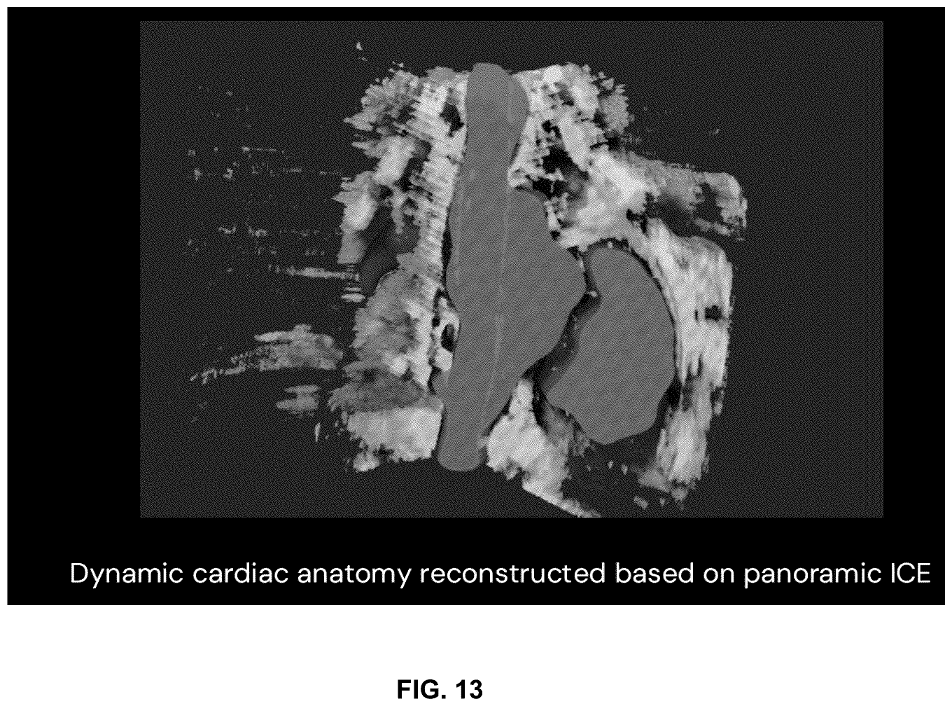

FIGS. 10-13 illustrate panoramic segmentation according to some embodiments of the systems and methods of the invention. In particular, FIGS. 10-13 illustrate examples of cardiac anatomy reconstructed based on panoramic ICE segmentation. FIG. 10 illustrates right atrium (RA) and left atrium (LA) segmentation based on ICE imaging with tracked tools. FIG. 11 illustrates RA segmentation based on 3D panoramic ICE imaging. FIG. 12 illustrates four-chamber segmentation based on 3D panoramic ICE imaging. FIG. 13 illustrates dynamic cardiac anatomy reconstructed based on panoramic ICE imaging, according to one embodiment of the systems and methods of the invention.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions, or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for interactive reconstruction of a digital anatomical model, the system comprising:
a console configured to be operably associated with an ultrasound imaging device and exchange data therewith, the console comprising a hardware processor coupled to non-transitory, computer-readable memory containing instructions executable by the processor to cause the console to:
receive data associated with at least one of cardiac and vascular anatomy, the data comprising catheter-based ultrasound imaging data and pulse phase data;
process and combine the received data; and
reconstruct, based on said processing and combining of data, an interactive digital model of an imaged anatomy, wherein the digital model encapsulates a representation of the anatomy in a spatial topology for live visualization of one or more anatomical regions of interest.

2. The system of claim 1, wherein the spatial topology comprises a topological map, wherein each point in the topological map represents tissue and/or one or more specific anatomical properties, wherein ultrasound imaging data from one or more directions and/or one or more views are combined to provide the representation of the anatomy.

3. The system of claim 1, wherein the digital model is at least one of a panoramic image reconstruction, a segmented surface model, and a mesh.

4. The system of claim 1, wherein reconstructing the digital model comprises image-based registration and/or simultaneous localization and mapping (SLAM).

5. The system of claim 1, wherein the catheter-based ultrasound imaging data is received from an intracardiac echocardiogram (ICE) 2D and/or 3D catheter.

6. The system of claim 5, wherein the ultrasound imaging data is received from a catheter trajectory used to image the anatomical region of interest in the catheter field-of-view in a plurality of temporally overlapping segments.

7. The system of claim 5, wherein the ultrasound imaging data is received from a plurality of cylindrical fields-of-view.

8. The system of claim 7, wherein the plurality of cylindrical fields-of-view are acquired via a rotating transducer array and/or a cylindrical folded transducer matrix.

9. The system of claim 5, wherein the ultrasound imaging data is received from a field of view whereby a motion of the catheter is perturbed from a smooth trajectory to change the viewing angle.

10. The system of claim 1, wherein the pulse phase data is one or more of electrocardiography (ECG) data, pulse oximetry data, and/or image-based intracardiac activity data.

11. The system of claim 1, wherein the received data further comprises three-dimensional (3D) position data.

12. The system of claim 11, wherein the position data comprises a spatial position and an orientation of a catheter and/or an interventional tool.

13. The system of claim 12, wherein the position data is 3D pose data comprising six degrees of freedom.

14. The system of claim 11, wherein the position data is obtained through one or more of electro-magnetic (EM) tracking data, impedance tracking data, image-based tracking data, and fiber optic shape sensing.

15. The system of claim 14, wherein combining the received data comprises selecting a 3D ultrasound image in the catheter-based ultrasound imaging data and mapping the 3D ultrasound image to a physical patient coordinate system using the 3D position data such that a registration of each 3D ultrasound image in the physical patient's coordinate system is initialized.

16. The system of claim 15, wherein combining the received data further comprises continuously registering subsequent 3D ultrasound images within the physical patient's coordinate system.

17. The system of claim 16, wherein the continuously registered subsequent 3D ultrasound images are partly overlapping such that the continuously registered and partly overlapping subsequent 3D ultrasound images are fused into one panoramic digital model.

18. The system of claim 17, wherein the panoramic digital model is updated sequentially over time as the catheter is moved through the one or more anatomical regions of interest.

19. The system of claim 17, wherein the panoramic digital model is segmented using a deep learning algorithm.

20. The system of claim 16, wherein continuous image registration comprises one or more of a rigid registration scheme and a deformable registration scheme.

21. The system of claim 16, wherein continuous 3D ultrasound image registration comprises filtering, using pulse phase-gating, the subsequent 3D ultrasound images along the catheter's trajectory, wherein a separate digital model is generated for each cardiac phase, and wherein 3D ultrasound images of a same cardiac phase are registered against a respective previous 3D ultrasound image and/or a fused 3D image.

22. The system of claim 21, wherein the filtered subsequent 3D ultrasound images are segmented using a supervised or an unsupervised deep-learning algorithm.

23. The system of claim 22, wherein reconstructing the digital model comprises fusing the segmented 3D ultrasound images using the registration results to produce a patient-specific digital 3D anatomical model.

24. The system of claim 23, wherein the patient-specific digital 3D anatomical model is continuously updated with subsequent registration and segmentation results.

25. The system of claim 24, wherein the continuously updated patient-specific digital 3D anatomical model is visualized simultaneously with the fused ultrasound image data to enable 3D navigation of the catheter.

26. The system of claim 25, wherein the separate 3D models for each cardiac phase are combined along the trajectory of the catheter to generate a temporal dynamic model that represents the patient-specific anatomy over time, wherein the dynamic model visualizes the heartbeat as a sequence of animated 3D models.

27. The system of claim 13, wherein the EM tracking data is combined with one or more previous registration results to predict a next 3D position.

28. The system of claim 17, wherein the predicted next 3D position is used for initialization of the continuous registration.

29. The system of claim 1, wherein the console is further operable to detect, localize, and segment one or more interventional tools within the 3D ultrasound images, wherein the segmented interventional tools are visualized in 3D within the digital model.

30. The system of claim 29, wherein detection, localization, and segmentation of the one or more interventional tools provides for contact assessment between a tool tip and a cardiac and/or a vascular wall.

31. The system of claim 1, wherein the interactive digital model is generated beginning from a catheter access site to one or more targets within a heart to provide an interactive visualization of patient-specific structure and/or morphology of intra-cardiac structures without the need for fluoroscopy.

32. The system of claim 31, wherein the intra-cardiac structures comprise one or more of trabeculae, tendons, chordae, and valves/leaflets.

33. The system of claim 1, wherein the console is further operable to automatically detect and localize anatomical landmarks for visualization within the digital model to aid navigation of the catheter and interventional tools.

34. The system of claim 1, wherein the catheter-based ultrasound imaging data is received from a catheter-based ultrasound comprising an endovascular and/or intravascular ultrasound configured for in-body applications.
